SEQUENCE LISTING (44 sequences)

SEQ ID NO: 1 - DNA, 25 bp, Synthetic construct
  source 1..25: mol_type = other DNA
gaattcctggattgtgtgtttaagt

SEQ ID NO: 2 - DNA, 24 bp, Synthetic construct
  source 1..24: mol_type = other DNA
cagaagggaactgtctagttcagt

SEQ ID NO: 3 - RNA, 50 nt, Synthetic construct
  source 1..50: mol_type = other RNA
cataattcttgtgtctactgtacagcatactgccgccagctggatttccc

SEQ ID NO: 4 - RNA, 53 nt, Synthetic construct
  source 1..53: mol_type = other RNA
gtgtctactgtacagcatactgccgccagctggatttgccaattctgagtaac

SEQ ID NO: 5 - RNA, 44 nt, Synthetic construct
  source 1..44: mol_type = other RNA
ggtgtcgaggagtactcctcgacaccctgccgccagctggattt

SEQ ID NO: 6 - RNA, 44 nt, Synthetic construct; identical to SEQ ID NO: 5
  source 1..44: mol_type = other RNA

SEQ ID NO: 7 - RNA, 44 nt, Synthetic construct; identical to SEQ ID NO: 5
  source 1..44: mol_type = other RNA

SEQ ID NO: 8 - RNA, 44 nt, Synthetic construct; identical to SEQ ID NO: 5
  source 1..44: mol_type = other RNA

SEQ ID NO: 9 - RNA, 44 nt, Synthetic construct; identical to SEQ ID NO: 5
  source 1..44: mol_type = other RNA

SEQ ID NO: 10 - RNA, 38 nt, Synthetic construct
  source 1..38: mol_type = other RNA
ggtgtcgaggcctcgacaccctgccgccagctggattt

SEQ ID NO: 11 - RNA, 38 nt, Synthetic construct; identical to SEQ ID NO: 10
  source 1..38: mol_type = other RNA

SEQ ID NO: 12 - RNA, 38 nt, Synthetic construct; identical to SEQ ID NO: 10
  source 1..38: mol_type = other RNA

SEQ ID NO: 13 - RNA, 38 nt, Synthetic construct; identical to SEQ ID NO: 10
  source 1..38: mol_type = other RNA

SEQ ID NO: 14 - RNA, 38 nt, Synthetic construct; identical to SEQ ID NO: 10
  source 1..38: mol_type = other RNA

SEQ ID NO: 15 - RNA, 38 nt, Synthetic construct
  source 1..38: mol_type = other RNA
ggtgccgaggcctcgacaccctgccgccagctggattt

SEQ ID NO: 16 - RNA, 37 nt, Synthetic construct
  source 1..37: mol_type = other RNA
ggtgcgaggcctggacaccctgccgccagctggattt

SEQ ID NO: 17 - RNA, 38 nt, Synthetic construct; identical to SEQ ID NO: 15
  source 1..38: mol_type = other RNA

SEQ ID NO: 18 - RNA, 38 nt, Synthetic construct; identical to SEQ ID NO: 15
  source 1..38: mol_type = other RNA

SEQ ID NO: 19 - RNA, 37 nt, Synthetic construct
  source 1..37: mol_type = other RNA
ggtgcgaggcctcgacaccctgccgccagctggattt

SEQ ID NO: 20 - RNA, 38 nt, Synthetic construct
  source 1..38: mol_type = other RNA
ggtgtcgaggcctggacaccctgccgccagctggattt

SEQ ID NO: 21 - RNA, 38 nt, Synthetic construct
  source 1..38: mol_type = other RNA
ggtgtcgaggcctgacaccctgccgccagcctggattt

SEQ ID NO: 22 - RNA, 38 nt, Synthetic construct; identical to SEQ ID NO: 20
  source 1..38: mol_type = other RNA

SEQ ID NO: 23 - DNA, 24 bp, Synthetic construct
  source 1..24: mol_type = other DNA
acctgggtatcactaaggtcttca

SEQ ID NO: 24 - DNA, 26 bp, Synthetic construct
  source 1..26: mol_type = other DNA
gttcaatcattaagaagacaaagggt

SEQ ID NO: 25 - RNA, 53 nt, Synthetic construct
  source 1..53: mol_type = other RNA
ggccccagcagcttcagtccctttctcgtcgatggtcagcacagccttatgca

SEQ ID NO: 26 - RNA, 50 nt, Synthetic construct; identical to SEQ ID NO: 3
  source 1..50: mol_type = other RNA

SEQ ID NO: 27 - RNA, 44 nt, Synthetic construct
  source 1..44: mol_type = other RNA
ggtgtcgaggagtactcctcgacaccacagcctcatgcacggcc

SEQ ID NO: 28 - RNA, 44 nt, Synthetic construct; identical to SEQ ID NO: 27
  source 1..44: mol_type = other RNA

SEQ ID NO: 29 - RNA, 44 nt, Synthetic construct; identical to SEQ ID NO: 27
  source 1..44: mol_type = other RNA

SEQ ID NO: 30 - RNA, 44 nt, Synthetic construct; identical to SEQ ID NO: 27
  source 1..44: mol_type = other RNA

SEQ ID NO: 31 - RNA, 44 nt, Synthetic construct; identical to SEQ ID NO: 27
  source 1..44: mol_type = other RNA

SEQ ID NO: 32 - RNA, 38 nt, Synthetic construct
  source 1..38: mol_type = other RNA
ggtgtcgaggcctcgacaccacagcctcatgcacggcc

SEQ ID NO: 33 - RNA, 38 nt, Synthetic construct; identical to SEQ ID NO: 32
  source 1..38: mol_type = other RNA

SEQ ID NO: 34 - RNA, 38 nt, Synthetic construct; identical to SEQ ID NO: 32
  source 1..38: mol_type = other RNA

SEQ ID NO: 35 - RNA, 38 nt, Synthetic construct; identical to SEQ ID NO: 32
  source 1..38: mol_type = other RNA

SEQ ID NO: 36 - RNA, 38 nt, Synthetic construct; identical to SEQ ID NO: 32
  source 1..38: mol_type = other RNA

SEQ ID NO: 37 - RNA, 38 nt, Synthetic construct
  source 1..38: mol_type = other RNA
ggtgccgaggcctcgacaccacagcctcatgcacggcc

SEQ ID NO: 38 - RNA, 38 nt, Synthetic construct
  source 1..38: mol_type = other RNA
ggtgtcgaggcctggacaccacagcctcatgcacggcc

SEQ ID NO: 39 - RNA, 38 nt, Synthetic construct; identical to SEQ ID NO: 37
  source 1..38: mol_type = other RNA

SEQ ID NO: 40 - RNA, 38 nt, Synthetic construct; identical to SEQ ID NO: 37
  source 1..38: mol_type = other RNA

SEQ ID NO: 41 - RNA, 38 nt, Synthetic construct; identical to SEQ ID NO: 37
  source 1..38: mol_type = other RNA

SEQ ID NO: 42 - RNA, 38 nt, Synthetic construct; identical to SEQ ID NO: 38
  source 1..38: mol_type = other RNA

SEQ ID NO: 43 - RNA, 38 nt, Synthetic construct; identical to SEQ ID NO: 38
  source 1..38: mol_type = other RNA

SEQ ID NO: 44 - RNA, 38 nt, Synthetic construct; identical to SEQ ID NO: 38
  source 1..38: mol_type = other RNA

(19) United States
(12) Patent Application Publication
(10) Pub. No.: US 2026/0242788 A1
(43) Pub. Date: Aug. 20, 2026

(54) Oligonucleotides for RNA Editing

(71) Applicant: KORRO BIO, INC., Cambridge, MA (US)

(72) Inventor: Mallikarjuna Reddy Putta, Cambridge, MA (US)

(21) Appl. No.: 19/161,179

(22) PCT Filed: Feb. 29, 2024
(86) PCT No.: PCT/US24/17792

§ 371 Date: Aug. 29, 2025

Related U.S. Application Data
(60) Provisional application No. 63/449,402, filed on Mar. 2, 2023.

(51) Int. Cl. C12N 15/113

(52) U.S. Cl. CPC C12N 15/113; C12N 2310/11; C12N 2310/315; C12N 2310/321; C12N 2310/322; C12N 2310/3231; C12N 2310/3341; C12N 2320/34

(57) ABSTRACT

The present invention relates to methods and compositions for editing a polynucleotide, e.g., a polynucleotide comprising a SNP associated with a disease or disorder.

The contents of the text file submitted electronically herewith are incorporated herein by reference in their entirety: A computer readable XML format copy of the Sequence Listing (Filename: “41029_SeqListing.xml”; Date created: Feb. 28, 2024; File size: 40,340 bytes).

### BACKGROUND

Adenosine deaminases acting on RNA (ADAR) are enzymes that bind to double-stranded RNA (dsRNA) and convert adenosine to inosine through deamination. In RNA, inosine functions similarly to guanosine for translation and replication. Thus, conversion of adenosine to inosine in an mRNA can result in a codon change that may lead to changes to the encoded protein and its functions. There are three known ADAR proteins expressed in humans, ADAR1, ADAR2, and ADAR3. ADAR1 and ADAR2 are expressed throughout the body whereas ADAR3 is expressed only in the brain. ADAR1 and ADAR2 are catalytically active, while ADAR3 is thought to be inactive.

Synthetic oligonucleotides have been shown capable of utilizing the ADAR proteins to edit target RNAs by deaminating particular adenosines in the target RNA. The oligonucleotides are complementary to the target RNA with the exception of at least one mismatch opposite the adenosine to be deaminated. Improved oligonucleotides, and complexes thereof, capable of utilizing the ADAR proteins to selectively edit target RNAs in a therapeutically effective manner are needed.

### SUMMARY

Described herein are duplex antisense oligonucleotides (duplex ASOs) comprising an activity region and a duplex region. In some embodiments, the duplex region comprises a 5′ portion and a 3′ portion, wherein the 5′ portion and the 3′ portion of the duplex region are substantially reverse complementary to one another. In some embodiments, at physiological conditions, the duplex regions of two oligonucleotides form a double-stranded region, allowing for generation of a complex comprising two oligonucleotides. In some embodiments, the two oligonucleotides in a complex are the same. Such a complex comprising two oligonucleotides allows them to anneal to each other based on their duplex regions. Generation of such a complex from two oligonucleotides allows for generation of a double-stranded region, which can allow for recruitment of ADAR proteins to effect ADAR editing. Generation of a complex with two identical oligonucleotides can obviate a need to synthesize and manufacture two different oligonucleotides to form a double-stranded complex.

Further, the length of each oligonucleotides comprised in a complex of two oligonucleotides formed by hybridization of two duplex regions to prepare a double-stranded region can be significantly shorter than a single oligonucleotide with a secondary structure allowing for a double-stranded region (for example, a single oligonucleotide designed to create a hairpin at physiological conditions). In this way, oligonucleotides designed to assemble into a complex with another oligonucleotide to prepare a double-stranded region can be cheaper and easier to manufacture as compared to oligonucleotides designed to generate a hairpin secondary structure.

Embodiment 1. An oligonucleotide comprising an activity region and a duplex region, wherein
the activity region comprises the structure:

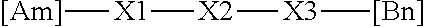

wherein each of A and B is a nucleotide;
m and n are each, independently, an integer from 2 to 50;
X¹, X², and X³ are each, independently, a nucleotide; and the duplex region consists of 8-30 nucleotides, wherein the nucleobase sequence of the duplex region is substantially reverse complementary to itself.

Embodiment 2. The oligonucleotide of embodiment 1, wherein the duplex region comprises 15-30, 14-29, 13-28, 12-27, 11-26, 10-25, 9-24, 8-23, 12-28, 13-27, 14-26, 15-25, 16-24, 17-23, 18-22, or 19-21 nucleotides.

Embodiment 3. The oligonucleotide of embodiment 1 or embodiment 2, wherein the activity region comprises 15-30, 14-29, 13-28, 12-27, 11-26, 10-25, 9-24, 8-23, 12-28, 13-27, 14-26, 15-25, 16-24, 17-23, 18-22, or 19-21 nucleotides.

Embodiment 4. The oligonucleotide of any one of embodiments 1-3, wherein the duplex region is 5′ of the activity region.

Embodiment 5. The oligonucleotide of any one of embodiments 1-3, wherein the duplex region is 3′ of the activity region.

Embodiment 6. The oligonucleotide of any one of embodiments 1-5, wherein the nucleotides of the duplex region are each independently selected from a 2′—O-C1-C6 alkyl-nucleotide, a 2′-amino-nucleotide, an arabino nucleic acid-nucleotide, a bicyclic-nucleotide, a 2′-O-methoxyethyl-nucleotide, a constrained ethyl (cEt)-nucleotide, a LNA-nucleotide, a ribonucleotide, and a DNA-nucleotide.

Embodiment 7. The oligonucleotide of any one of embodiments 1-6, wherein at physiological conditions, the duplex regions of two oligonucleotides form a double-stranded region.

Embodiment 8. The oligonucleotide of embodiment 7, wherein the double-stranded region formed by the duplex region of two oligonucleotides is 8-30 base pairs long, including any mismatches.

Embodiment 9. The oligonucleotide of embodiment 7 or embodiment 8, wherein the double-stranded region formed by the duplex region of two oligonucleotides comprises 0, 1, 2, 3, 4, or 5 mismatches.

Embodiment 10. The oligonucleotide of any one of embodiments 7-9, wherein the double-stranded region formed by the duplex regions of two oligonucleotides comprises 11-30 base pairs or 18-26 base pairs, including any mismatches.

Embodiment 11. The oligonucleotide of any one of embodiments 7-10, wherein the double-stranded regions formed by the duplex regions of two oligonucleotides is capable of recruiting an adenosine deaminase acting on RNA (ADAR) enzyme.

Embodiment 12. The oligonucleotide of any one of embodiments 1-11, wherein the oligonucleotide consists of 20-80 nucleotides, or 20-70 nucleotides, or 20-60 nucleotides, or 30-60 nucleotides, or 30-50 nucleotides.

Embodiment 13. The oligonucleotide of any one of embodiments 1-12, wherein the activity region is substantially complementary to a target mRNA.

Embodiment 14. The oligonucleotide of any one of embodiments 1-13, wherein at least one nucleotide of A and/or B is a 2′-F-nucleotide, optionally wherein at least one 2′-F-nucleotide is at a position selected from +8, +3, −3, −7, −19 and −22, wherein X² is position 0, X¹ is position −1, and X³ is position +1.

Embodiment 15. The oligonucleotide of any one of embodiments 1-14, wherein all purines comprised in the duplex region are 2′-fluoro nucleotides and all pyrimidines comprised in the duplex region are 2′-O-methoxyethyl nucleotides.

Embodiment 16. The oligonucleotide of any one of embodiments 14-15, wherein the remaining nucleotides of [Aₘ] are each independently selected from a 2′-O—C₁-C₆ alkyl-nucleotide, a 2′-amino-nucleotide, an arabino nucleic acid-nucleotide, a bicyclic-nucleotide, a 2′-O-methoxyethyl-nucleotide, a constrained ethyl (cEt)-nucleotide, a LNA-nucleotide, a ribonucleotide, and a DNA-nucleotide.

Embodiment 17. The oligonucleotide of any one of embodiments 14-15, wherein the remaining nucleotides of [Aₘ] are each independently selected from a 2′-O-methyl-nucleotide, a 2′-F-nucleotide, a 2′-O-methoxyethyl-nucleotide, a cEt-nucleotide, a LNA-nucleotide, a ribonucleotide, and a DNA-nucleotide.

Embodiment 18. The oligonucleotide of any one of embodiments 14-15, wherein the remaining nucleotides of [Aₘ] are 2′-O-methyl-nucleotides.

Embodiment 19. The oligonucleotide of any one of embodiments 1-18, wherein [Aₘ] comprises at least one phosphorothioate linkage.

Embodiment 20. The oligonucleotide of any one of embodiments 1-19, wherein [Aₘ] comprises at least four terminal phosphorothioate linkages.

Embodiment 21. The oligonucleotide of any one of embodiments 19-20, wherein at least one phosphorothioate linkage is stereopure.

Embodiment 22. The oligonucleotide of any one of embodiments 1-21, wherein [Bₙ] comprises at least one nuclease resistant nucleotide.

Embodiment 23. The oligonucleotide of any one of embodiments 1-22, wherein each nucleotide of [Bₙ] is a nuclease resistant nucleotide.

Embodiment 24. The oligonucleotide of any one of embodiments 1-23, wherein each nucleotide of [Bₙ] is independently selected from a 2′-O—C₁-C₆ alkyl-nucleotide, a 2′-amino-nucleotide, an arabino nucleic acid-nucleotide, a bicyclic-nucleotide, a 2′-O-methoxyethyl-nucleotide, a constrained ethyl (cEt)-nucleotide, a LNA-nucleotide, a ribonucleotide, and a DNA-nucleotide.

Embodiment 25. The oligonucleotide of any one of embodiments 1-24, wherein each nucleotide of [Bₙ] is independently selected from a 2′-O-methyl-nucleotide, a 2′-O-methoxyethyl-nucleotide, a cEt-nucleotide, a LNA-nucleotide, a ribonucleotide, and a DNA-nucleotide.

Embodiment 26. The oligonucleotide of any one of embodiments 1-25, wherein each nucleotide of [Bₙ] is a 2′-O-methyl-nucleotide.

Embodiment 27. The oligonucleotide of any one of embodiments 1-26, wherein [Bₙ] comprises at least one phosphorothioate linkage.

Embodiment 28. The oligonucleotide of any one of embodiments 1-27, wherein [Bₙ] comprises at least four terminal phosphorothioate linkages.

Embodiment 29. The oligonucleotide of any one of embodiments 27-28, wherein at least one phosphorothioate linkage is stereopure.

Embodiment 30. The oligonucleotide of any one of embodiments 1-29, wherein the oligonucleotide comprises 1, 2, 3, or 4 2′-F-nucleotides.

Embodiment 31. The oligonucleotide of any one of embodiments 1-30, wherein X2 is not a 2′-O-methyl-nucleotide.

Embodiment 32. The oligonucleotide of any one of embodiments 1-31, wherein X¹, X², and X³ are not 2′-O-methyl-nucleotides.

Embodiment 33. The oligonucleotide of any one of embodiments 1-32, wherein X¹, X², and X³ are 2′-deoxyribonucleotides.

Embodiment 34. The oligonucleotide of any one of embodiments 1-33, wherein X² comprises a cytosine or 5-methylcytosine nucleobase.

Embodiment 35. The oligonucleotide of embodiment 34, wherein X² comprises a cytosine nucleobase.

Embodiment 36. The oligonucleotide of any one of embodiments 1-35, wherein the oligonucleotide further comprises a 5′-cap structure.

Embodiment 37. The oligonucleotide of any one of embodiments 1-36, wherein the oligonucleotide comprises at least one alternative nucleobase.

Embodiment 38. The oligonucleotide of any one of embodiments 1-37, wherein m is 5 to 25.

Embodiment 39. The oligonucleotide of any one of embodiments 1-38, wherein n is 5 to 25.

Embodiment 40. The oligonucleotide of any one of embodiments 1-39, wherein the activity region is at least 70%, at least 75%, at least 80%, at least 85%, at least 90%, or at least 95% complementary to a target mRNA.

Embodiment 41. The oligonucleotide of any one of embodiments 1-40, wherein the activity region is complementary to a target mRNA comprising a single nucleotide polymorphism (SNP) associated with a disease or disorder.

Embodiment 42. The oligonucleotide of embodiment 1-41, wherein the target mRNA encodes a protein comprising a pathogenic amino acid resulting from the SNP.

Embodiment 43. The oligonucleotide of any one of embodiments 1-42, wherein the oligonucleotide is capable of effecting an ADAR-mediated adenosine to inosine alteration of an adenosine in a target mRNA, wherein X² aligns with the adenosine in the target mRNA to be altered to an inosine.

Embodiment 44. A complex of two oligonucleotides of any one of embodiments 1-43.

Embodiment 45. The complex of embodiment 44, wherein the two oligonucleotides are the same.

Embodiment 46. The complex of embodiment 44 or embodiment 45, wherein the complex comprises a double-stranded region formed by the duplex regions of the two oligonucleotides.

Embodiment 47. The complex of any one of embodiments 44-46, comprising a target mRNA.

Embodiment 48. The complex of embodiment 47, wherein the activity region hybridizes to the target mRNA.

Embodiment 49. A method of editing a target polynucleotide, comprising contacting the target polynucleotide with the oligonucleotide of any one of embodiments 1-43 or the complex of any one of embodiments 44-48, thereby editing the polynucleotide.

Embodiment 50. The method of embodiment 49, wherein the target polynucleotide is contacted with the oligonucleotide in a cell.

Embodiment 51. The method of embodiment 50, wherein the cell endogenously expresses ADAR.

Embodiment 52. The method of embodiment 51, wherein the ADAR is a human ADAR.

Embodiment 53. The method of embodiment 51, wherein the ADAR is human ADAR1.

Embodiment 54. The method of embodiment 51, wherein the ADAR is human ADAR2.

Embodiment 55. The method of any one of embodiments 49-54, wherein the cell is selected from eukaryotic cell, a mammalian cell, and a human cell.

Embodiment 56. The method of any one of embodiments 55, wherein the cell is in vivo.

Embodiment 57. The method of any one of embodiments 55, wherein the cell is ex vivo.

Embodiment 58. A method of treating a disease or disorder associated with a single nucleotide polymorphism (SNP) in a subject in need thereof, comprising administering to the subject the oligonucleotide of any one of embodiments 1-43 or the complex of any one of embodiments 44-48.

Embodiment 59. The method of embodiment 58, wherein the oligonucleotide or the complex is capable of effecting an ADAR-mediated adenosine to inosine alteration of the SNP associated with the disease or disorder, thereby treating the disease or disorder.

Embodiment 60. The method of any one of embodiments 58-59, wherein the subject is a human subject.

Embodiment 61. The method of any one of embodiments 58-60, wherein the target mRNA encodes a protein comprising a pathogenic amino acid resulting from the SNP.

Embodiment 62. The method of embodiment 61, wherein the adenosine to inosine alteration substitutes the pathogenic amino acid with a wildtype amino acid.

Embodiment 63. The method of embodiment 61, wherein the adenosine is substituted for another nucleotide that replaces the pathogenic amino acid with an amino acid that confers the substantially similar protein activity as the wildtype amino acid.

Embodiment 64. The embodiment of claim 63, wherein adenosine is substituted for another nucleotide that replaces the pathogenic amino acid with an amino acid that confers restored or modulated function as compared to a protein comprising the pathogenic amino acid.

### BRIEF DESCRIPTION OF THE DRAWINGS

FIGS. 1A and 1B show a duplex ASO (i.e., an ASO comprising a duplex region as described herein (A) and an ASO comprising a GluR hairpin region (B). As shown in (A), two ASOs can generate a complex comprising two oligonucleotides annealed together to form a double-stranded region, wherein the duplex region of a first oligonucleotide is oriented 5′ to 3′ and annealed to the duplex region of a second oligonucleotide that is oriented 3′ to 5′. This double-stranded region can allow for recruitment of ADAR proteins to effect ADAR editing.

### DETAILED DESCRIPTION

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including, but not limited to”.

The term “or” is used herein to mean, and is used interchangeably with, the term “and/or,” unless context clearly indicates otherwise.

The term “about” is used herein to mean within the typical ranges of tolerances in the art, e.g., acceptable variation in time between doses, acceptable variation in dosage unit amount. For example, “about” can be understood as within about 2 standard deviations from the mean. In certain embodiments, about means+10%. In certain embodiments, about means+5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, an oligonucleotide with “no more than 5 unmodified nucleotides” has 5, 4, 3, 2, 1, or 0 unmodified nucleotides. When “no more than” is present before a series of numbers or a range, it is understood that “no more than” can modify each of the numbers in the series or range.

As used herein, the term “single nucleotide polymorphisms (SNP),” refers to a variation at a single position in a DNA sequence among individuals. If more than 1% of a population does not carry the same nucleotide at a specific position in the DNA sequence, then this variation can be classified as a SNP. If a SNP occurs within a gene, then the gene is described as having more than one allele. In these cases, SNPs may lead to variations in the amino acid sequence. For example, at a specific base position in the human genome, the C nucleotide can appear in most individuals, but in a minority of individuals, the position is occupied by an A. This means that there is a SNP at this specific position, and the two possible nucleotide variations, C or A, are the two alleles for this position.

SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions (e.g., 5′UTR) can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant is a variation in a single nucleotide without any limitations of frequency and can arise in somatic cells. A somatic single nucleotide variation can also be called a single-nucleotide alteration.

Although a particular SNP may not cause a disorder, some SNPs are associated with certain diseases. These associations allow for the use of specific SNPs to evaluate an individual's genetic predisposition to develop a disease. In addition, if certain SNPs are known to be associated with a trait, then examination of certain stretches of DNA near these SNPs will help identify the gene or genes responsible for the trait.

As used herein, the phrase “SNP associated with a disease or disorder” refers to any SNPs that cause a particular disease or disorder. Exemplary SNPs associated with a disease or disorder include but are not limited to, any single nucleotide changes that result in the presence of a pathogenic amino acid in the encoded protein.

The term “pathogenic amino acid” refers to any amino acid that is not a wildtype amino acid in a protein and which leads to a pathogenesis.

The terms “pathogenic mutation”, “pathogenic variant”, “disease causing mutation”, “disease causing variant”, or “deleterious mutation”, refers to a genetic alteration or mutation that increases an individual's susceptibility or predisposition to a certain disease or disorder. In some embodiments, the pathogenic mutation comprises at least one wildtype amino acid substituted by at least one pathogenic amino acid in a protein encoded by a gene.

The term “adenosine deaminase”, as used herein, refers to a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). In some embodiments, the adenosine deaminase catalyzes the hydrolytic deamination of adenine or adenosine in ribonucleic acid (RNA). The adenosine deaminases may be from any organism, such as a human, chimpanzee, gorilla, monkey, cow, dog, rat, or mouse. In some embodiments, the adenosine deaminase is from a bacterium, such as E. coli, S. aureus, S. typhi, S. putrefaciens, H. influenzae, or C. crescentus. In some embodiments, the deaminase or deaminase domain is a variant of a naturally occurring deaminase from an organism, such as a human, chimpanzee, gorilla, monkey, cow, dog, rat, or mouse. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to a naturally occurring deaminase. For example, deaminase domains are described in International PCT Application Nos. PCT/2017/045381 (WO 2018/027078) and PCT/US2016/058344 (WO 2017/070632), each of which is incorporated herein by reference for its entirety. Also see Komor, A. C., et al., Nature 533, 420-424 (2016); Gaudelli, N. M., et al., Nature 551, 464-471 (2017); Komor, A. C., et al., Science Advances 3:eaao4774 (2017), and Rees, H. A., et al., Nat Rev Genet. 2018; 19(12):770-788. doi: 10.1038/s41576-018-0059-1, the entire contents of which are hereby incorporated by reference.

As used herein, the term “Adenosine deaminases acting on RNA (ADAR)” refers to editing enzymes which can recognize certain structural motifs of double-stranded RNA (dsRNA), bind to dsRNA and convert adenosine to inosine through deamination, resulting in recoding of amino acid codons that may lead to changes to the encoded protein and its function. The nucleobases surrounding the editing site, especially the one immediately 5′ of the editing site and one immediately 3′ to the editing site, which together with the editing site are termed the triplet, play an important role in the deamination of adenosine. A preference for U at the 5′ position and G at the 3′ position relative to the editing site, was revealed from the analysis of yeast RNAs efficiently edited by overexpressed human ADAR2 and ADAR1. (See Wang et al., (2018) Biochemistry, 57: 1640-1651; Eifler et al., (2013) Biochemistry, 52: 7857-7869, and Eggington et al., (2011) Nat. Commun., 319: 1-9.) There are three known ADAR proteins expressed in humans, ADAR1, ADAR2, and ADAR3. ADAR1 and ADAR2 are expressed throughout the body, whereas ADAR3 is expressed only in the brain. ADAR1 and ADAR2 are catalytically active, while ADAR3 is thought to be inactive. Recruiting ADAR to specific sites of selected transcripts and deamination of adenosine regardless of neighboring bases holds great promise for the treatment of disease.

As used herein, the term “duplex region” refers to a region of an oligonucleotide the comprises a 5′ portion and a 3′ portion that are substantially reverse complementary to one another, such that duplex regions of two copies of the oligonucleotide are capable of hybridizing to form a double-stranded region. At physiological conditions, the duplex regions of two single-stranded oligonucleotides (wherein the two oligonucleotides comprise the same or substantially the same duplex region) can form a double-stranded region. An oligonucleotide comprising a duplex region (i.e. a region of substantially reverse complementary sequence) may be termed a “palindrome” or “palindromic oligonucleotide,” and such a palindromic oligonucleotide may be capable of hybridizing to another palindromic oligonucleotide with the same or substantially similar duplex region. This double-stranded region is comprised within a complex of two oligonucleotides described herein, wherein the duplex region of a first oligonucleotide is oriented 5′ to 3′ and annealed to the duplex region of a second oligonucleotide that is oriented 3′ to 5′. Accordingly, the duplex regions described herein are designed to mediate intermolecular association of two oligonucleotides, resulting in a double-stranded region.

As used herein, the term “reverse complementary,” refers to sequence wherein the 5′ portion and the 3′ portion of said sequence comprise sequences that could hybridize to each other under certain conditions. A reverse complementary sequence may be comprised in a duplex region of the instant invention. In a reverse complementary sequence, a 5′ sequence oriented 5′ to 3′ can hybridize with a 3′ sequence oriented 3′ to 5′. Reverse complementary sequences may allow for formation of a complex of two oligonucleotides, wherein the reverse complementary sequences comprised in the duplex regions of the two oligonucleotides can hybridize to each other. Reverse complementary sequences include base-pairing of the duplex regions of two oligonucleotides over the entire length of one or both nucleotide sequences of the duplex regions. Such sequences can be referred to as “fully reverse complementary” with respect to each other herein. However, where a first sequence is referred to as “substantially reverse complementary” with respect to a second sequence herein, the two sequences can be fully reverse complementary, or they can form one or more, but generally no more than 5, 4, 3 or 2 mismatched base pairs upon hybridization for a double-stranded region up to 30 base pairs. The binding of two duplex regions would generally have a higher affinity with a greater number of reverse complementary nucleotides. In this way, a duplex region of one oligonucleotide may preferentially form a duplex with the duplex region of another oligonucleotide instead of forming a secondary structure within the oligonucleotide, such as a hairpin.

As used herein, the term “activity region” refers to a region of an oligonucleotide that mediates a desired activity. In some embodiments, the activity region is complementary to a target mRNA, and is capable of mediating ADAR-dependent mRNA editing.

As used herein, the term “ADAR-recruiting domain” refers to a domain of an oligonucleotide that is able to recruit an ADAR enzyme. For example, such recruiting domains may be double-stranded regions that act as recruitment and binding regions for the ADAR enzyme. The double-stranded regions formed by the hybridization of the duplex regions of two oligonucleotides as described herein may form or comprise an ADAR recruiting domain. The ADAR-recruiting domain portion may act to recruit an endogenous ADAR enzyme present in the cell. Such ADAR-recruiting domains do not require conjugated entities or presence of modified recombinant ADAR enzymes. Alternatively, the ADAR-recruiting portion may act to recruit a recombinant ADAR fusion protein that has been delivered to a cell or to a subject via an expression vector construct including a polynucleotide encoding an ADAR fusion protein. Such ADAR-fusion proteins may include the deaminase domain of ADAR1 or ADAR2 enzymes fused to another protein, e.g., to the MS2 bacteriophage coat protein. An ADAR-recruiting domain may be a nucleotide sequence based on a natural substrate (e.g., the GluR2 receptor pre-mRNA; such as a GluR2 ADAR-recruiting domain), a Z-DNA structure, or a domain known to recruit another protein which is part of an ADAR fusion protein, e.g., an MS2 ADAR-recruiting domain known to be recognized by the dsRNA binding regions of ADAR. An ADAR-recruiting domain can be a double-stranded region, formed by annealing of the duplex regions of two separate oligonucleotides, wherein a first oligonucleotide has a duplex region oriented 5′ to 3′ and a second oligonucleotide anneals via its duplex region oriented 3′ to 5′.

As used herein, the term “Z-DNA” refers to a left-handed conformation of the DNA double helix or RNA stem loop structures. Such DNA or dsRNA helices wind to the left in a zigzag pattern (as opposed to the right, like the more commonly found B-DNA form). Z-DNA is a known high-affinity ADAR binding substrate and has been shown to bind to human ADAR1 enzyme.

“G,” “C,” “A,” “T,” and “U” each generally stand for a naturally-occurring nucleotide that contains guanine, cytosine, adenine, thymidine, and uracil as a base, respectively. However, it will be understood that the term “nucleotide” can also refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide including a nucleotide bearing such replacement moiety. For example, without limitation, a nucleotide including hypoxanthine as its base can base pair with nucleotides containing adenine, cytosine, or uracil. Hence, nucleotides containing uracil, guanine, or adenine can be replaced in the nucleotide sequences of oligonucleotides featured in the invention by a nucleotide containing, for example, hypoxanthine. In another example, adenine and cytosine anywhere in the oligonucleotide can be replaced with guanine and uracil, respectively to form G-U wobble base pairing with the target mRNA. Sequences containing such replacement moieties are suitable for the compositions and methods featured in the invention.

The terms “nucleobase” and “base” include the purine (e.g., adenine and guanine) and pyrimidine (e.g., uracil, thymine, and cytosine) moiety present in nucleosides and nucleotides which form hydrogen bonds in nucleic acid hybridization. In the context of the present invention, the term nucleobase also encompasses alternative nucleobases which may differ from naturally-occurring nucleobases but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. As used herein, “an alternative nucleobase” may refer to any nucleobase that is not adenine, guanine, cytosine, thymidine, uracil, xanthine, or hypoxanthine. Such variants are, for example, described in Hirao et al (2012) Accounts of Chemical Research vol 45, page 2055 and Bergstrom (2009) Current Protocols in Nucleic Acid Chemistry Suppl. 37 Chapter 1, unit 4.1.

In a some embodiments, the nucleobase moiety may be a modified purine or pyrimidine, such as substituted purine or substituted pyrimidine, such as an “alternative nucleobase” selected from isocytosine, pseudoisocytosine, 5-methylcytosine, 5-thiozolo-cytosine, 5-propynyl-cytosine, 5-propynyl-uracil, 5-bromouracil, 5-thiazolo-uracil, 2-thio-uracil, pseudouracil, 1-methylpseudouracil, 5-methoxyuracil, 2′-thio-thymine, hypoxanthine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g. A, T, G, C, or U, wherein each letter may optionally include alternative nucleobases of equivalent function.

A “sugar” or “sugar moiety,” includes naturally occurring sugars having a furanose ring. A sugar also includes an “alternative sugar,” defined as a structure that is capable of replacing the furanose ring of a nucleoside. In certain embodiments, alternative sugars are non-furanose (or 4′-substituted furanose) rings or ring systems or open systems. Such structures include simple changes relative to the natural furanose ring, such as a six-membered ring, or may be more complicated as is the case with the non-ring system used in peptide nucleic acid. Alternative sugars may also include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization.

A “nucleotide,” as used herein refers to a monomeric unit of an oligonucleotide or polynucleotide that includes a nucleoside and an internucleoside linkage. The internucleoside linkage may or may not include a phosphate linkage. Similarly, “linked nucleosides” may or may not be linked by phosphate linkages. Many “alternative internucleoside linkages” are known in the art, including, but not limited to, phosphorothioate and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide” as used herein, refers to a nucleotide having an alternative nucleobase or an alternative sugar, and an internucleoside linkage, which may may be an alternative nucleoside linkage.

The term “nucleoside” refers to a monomeric unit of an oligonucleotide or a polynucleotide having a nucleobase and a sugar moiety. A nucleoside may include those that are naturally-occurring as well as alternative nucleosides, such as those described herein. The nucleobase of a nucleoside may be a naturally-occurring nucleobase or an alternative nucleobase. Similarly, the sugar moiety of a nucleoside may be a naturally-occurring sugar or an alternative sugar.

The term “alternative nucleoside” refers to a nucleoside having an alternative sugar or an alternative nucleobase, such as those described herein.

The term “nuclease resistant nucleotide” as used herein refers to nucleotides which limit nuclease degradation of oligonucleotides. Nuclease resistant nucleotides generally increase stability of oligonucleotides by being poor substrates for the nucleases. Nuclease resistant nucleotides are known in the art, e.g., 2′-O-methyl-nucleotides and 2′-fluoro-nucleotides.

The terms “oligonucleotide” and “polynucleotide” as used herein, are defined as it is generally understood by the skilled person as a molecule including two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention may be man-made, and is chemically synthesized, and is typically purified or isolated. Oligonucleotide is also intended to include (i) compounds that have one or more furanose moieties that are replaced by furanose derivatives or by any structure, cyclic or acyclic, that may be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that may be used as a point of covalent attachment for the base moiety. The oligonucleotide of the invention may include one or more alternative nucleosides or nucleotides (e.g., including those described herein). It is also understood that oligonucleotide includes compositions lacking a sugar moiety or nucleobase but is still capable of forming a pairing with or hybridizing to a target sequence.

“Oligonucleotide” refers to a short polynucleotide (e.g., of 100 or fewer linked nucleosides).

The phrases “an oligonucleotide that is capable of effecting an adenosine deaminase acting on RNA (ADAR)-mediated adenosine to inosine alteration” or “a complex that is capable of effecting an ADAR-mediated adenosine to inosine alteration” refer to an oligonucleotide or complex comprising two oligonucleotides that is specific for a target sequence and is capable to be utilized for the deamination reaction of a specific adenosine in a target sequence through an ADAR-mediated pathway. Each oligonucleotide may comprise a nucleic acid sequence complementary to a target sequence, e.g., an mRNA sequence comprising the SNP associated with a disease. In some embodiments, the oligonucleotides may comprise a nucleic acid sequence complementary to target mRNA with the exception of at least one mismatch. The oligonucleotide includes a mismatch opposite the target adenosine. In some embodiments, the oligonucleotides for use in the methods of the present invention do not include those used by any other gene editing technologies known in the art., e.g., CRISPR.

The oligonucleotide may be of any length, and may range from about 10-80 bases in length, e.g., about 15-80 bases in length, about 18-80 bases in length, about 20-80 bases in length, about 20-70 bases in length, about 20-60 bases in length, about 30-60 bases in length, or about 30-50 bases in length for example, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 bases in length, such as about 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-31, 15-30, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-31, 18-30, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-31, 19-30, 20-50, 20-49, 20-48, 20-47, 20-46, 20-45, 20-44,20-43, 20-42, 20-41, 20-40, 20-39, 20-38, 20-37, 20-36, 20-35, 20-34, 20-33, 20-32, 20-31, 20-31, 20-30, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-31, or 21-30 bases in length. Ranges and lengths intermediate to the above recited ranges and lengths are also contemplated to be part of the invention.

The term “linker” or “linking group” is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety to an oligonucleotide (e.g. the termini of region A or C). In some embodiments of the invention the conjugate or oligonucleotide conjugate of the invention may optionally, include a linker region which is positioned between the oligonucleotide and the conjugate moiety. In some embodiments, the linker between the conjugate and oligonucleotide is biocleavable. Phosphodiester containing biocleavable linkers are described in more detail in WO 2014/076195 (herein incorporated by reference).

“Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. Complementary sequences between an oligonucleotide and a target sequence as described herein, include base-pairing of the oligonucleotide or polynucleotide including a first nucleotide sequence to an oligonucleotide or polynucleotide including a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally no more than 5, 4, 3 or 2 mismatched base pairs upon hybridization for a duplex up to 30 base pairs, while retaining the ability to hybridize under the conditions most relevant to their ultimate application, e.g., deamination of an adenosine. “Substantially complementary” can also refer to a polynucleotide that is substantially complementary to a contiguous portion of the mRNA of interest (e.g., an mRNA having a target adenosine). For example, a polynucleotide is complementary to at least a part of the mRNA of interest if the sequence is substantially complementary to a non-interrupted portion of the mRNA of interest.

As used herein, and unless otherwise indicated, the term “complementary,” when used to describe a first nucleotide or nucleoside sequence in relation to a second nucleotide or nucleoside sequence, refers to the ability of an oligonucleotide or polynucleotide including the first nucleotide or nucleoside sequence to hybridize and form a duplex structure under certain conditions with an oligonucleotide or polynucleotide including the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides or nucleosides. In some embodiments, an oligonucleotide or portion of an oligonucleotide is at least 80%, at least 85%, at least 90%, at least 95%, at least 99%, or 100% complementary to a reference (e.g., target) sequence. In such embodiments, the percent complementarity is calculated over the length of the oligonucleotide or portion thereof.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). In some embodiments, the presently disclosed compositions can efficiently generate an “intended mutation”, such as a point mutation, in a nucleic acid (e.g., a nucleic acid within a genome of a subject) without generating a significant number of unintended mutations, such as unintended point mutations. In some embodiments, an intended mutation is a mutation that is generated by a specific guide oligonucleotide, specifically designed to generate the intended mutation. In general, mutations made or identified in a sequence (e.g., an amino acid sequence as described herein) are numbered in relation to a reference (or wildtype) sequence, i.e., a sequence that does not contain the mutations. The skilled practitioner in the art would readily understand how to determine the position of mutations in amino acid and nucleic acid sequences relative to a reference sequence.

The term “contacting,” as used herein, includes contacting a target mRNA by any means. In some embodiments, a target mRNA is contacted with an oligonucleotide in a cell. Contacting an mRNA in a cell with an oligonucleotide includes contacting the mRNA in a cell in vitro or in vivo with an oligonucleotide or complex.

Contacting a cell in vitro may be done, for example, by incubating the cell with the oligonucleotide or complex. Contacting a cell in vivo may be done, for example, by injecting the oligonucleotide or complex into or near the tissue where the cell is located, or by injecting the oligonucleotide or complex into another area, e.g., the bloodstream or the subcutaneous space, such that the agent will subsequently reach the tissue where the cell to be contacted is located. In some embodiments, a complex of two oligonucleotides is formed first, and then administered in vivo. For example, a complex of two oligonucleotides may be prepared in solution, and then administered to a subject. In some embodiments, the oligonucleotide or complex may contain and/or be coupled to a ligand that directs the oligonucleotide to a site of interest. Combinations of in vitro and in vivo methods of contacting are also possible. For example, a cell may also be contacted in vitro with an oligonucleotide or complex and subsequently transplanted into a subject.

In one embodiment, contacting a cell with an oligonucleotide or complex includes “introducing” or “delivering the oligonucleotide into the cell” by facilitating or effecting uptake or absorption into the cell. Absorption or uptake of an oligonucleotide or complex can occur through unaided diffusive or active cellular processes, or by auxiliary agents or devices. Introducing an oligonucleotide or complex into a cell may be in vitro and/or in vivo. For example, for in vivo introduction, oligonucleotides or complex can be injected into a tissue site or administered systemically. In vitro introduction into a cell includes methods known in the art such as electroporation and lipofection. Further approaches are described herein below and/or are known in the art.

As used herein, “lipid nanoparticle” or “LNP” is a vesicle including a lipid layer encapsulating a pharmaceutically active molecule, such as a nucleic acid molecule, e.g., an oligonucleotide. LNP refers to a stable nucleic acid-lipid particle. LNPs typically contain a cationic, ionizable lipid, a non-cationic lipid, and a lipid that prevents aggregation of the particle (e.g., a PEG-lipid conjugate). LNPs are described in, for example, U.S. Pat. Nos. 6,858,225; 6,815,432; 8,158,601; and 8,058,069, the entire contents of which are hereby incorporated herein by reference.

As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide composition. The lipophilic material isolates the aqueous interior from an aqueous exterior, which typically does not include the oligonucleotide composition, although in some examples, it may. Liposomes also include “sterically stabilized” liposomes, a term which, as used herein, refers to liposomes including one or more specialized lipids that, when incorporated into liposomes, result in enhanced circulation lifetimes relative to liposomes lacking such specialized lipids.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. Percent identity is calculated over the length of the shorter of the two sequences being compared.

By “level” is meant a level or activity of a protein or mRNA, as compared to a reference. The reference can be any useful reference, as defined herein. By a “decreased level” or an “increased level” of a protein or mRNA is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2-fold, about 3-fold, about 3.5-fold, about 4.5-fold, about 5-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, g/mL, ng/mL) or percentage relative to total protein or mRNA in a sample.

The term “pharmaceutical composition,” as used herein, represents a composition containing a compound described herein formulated with a pharmaceutically acceptable excipient, and preferably manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for intrathecal injection; for intracerebroventricular injections; for intraparenchymal injection; or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder; a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the term “administration” refers to the administration of a composition (e.g., a compound or a preparation that includes a compound as described herein) to a subject or system. Administration to an animal subject (e.g., to a human) may be by any appropriate route, such as the one described herein.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “treat,” “treated,” or “treating” mean therapeutic treatment wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment may include ameliorating one or more symptoms of a disorder as measurable, for example, by a clinician. Treatment also includes prolonging survival as compared to expected survival if not receiving treatment.

As used herein, the terms “effective amount,” “therapeutically effective amount,” and “a “sufficient amount” of an agent that results in a therapeutic effect (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating a disorder, it is an amount of the agent that is sufficient to achieve a treatment response as compared to the response obtained without administration. The amount of a given agent will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent is an amount which results in a beneficial or desired result in a subject as compared to a control. As defined herein, a therapeutically effective amount of an agent may be readily determined by one of ordinary skill by routine methods known in the art. In some embodiments, a therapeutically effective amount of an agent ameliorates one or more symptoms of a disorder as measurable, for example, by a clinician. Dosage regimen may be adjusted to provide the optimum therapeutic response.

A “therapeutically-effective amount” also includes an amount (either administered in a single or in multiple doses) of an oligonucleotide that produces some desired local or systemic effect at a reasonable benefit/risk ratio applicable to any treatment. Oligonucleotides employed in the methods of the present invention may be administered in a sufficient amount to produce a reasonable benefit/risk ratio applicable to such treatment.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control

The details of one or more embodiments of the invention are set forth in the description below. Other features, objects, and advantages of the invention will be apparent from the description and from the claims.

### II. Exemplary Methods

In some embodiments, the invention is used to make desired changes in a target sequence, e.g., a target sequence comprising a SNP associated with a disease, in a cell or a subject by site-directed editing of nucleotides through the use of an oligonucleotide that is capable of effecting an adenosine deaminase acting on RNA (ADAR)-mediated adenosine to inosine alteration of the SNP. As a result, the target sequence is edited through an adenosine deamination reaction mediated by ADAR, converting adenosines into inosine.

The ADAR mediated editing may be in 5′ or 3′ untranslated regions of a target RNA, in splice sites, in exons (changing amino acids in protein translated from the target RNA, changing codon usage or splicing behavior by changing exonic splicing silencers or enhancers, and/or introducing or removing start or stop codons), in introns (changing splicing by altering intronic splicing silencers or intronic splicing enhancers, branch points) and in general in any region affecting RNA stability, structure or functioning. The target RNA sequence may comprise a mutation that one may wish to correct or alter, such as a transition or a transversion.

RNA editing enzymes are known in the art. In some embodiments, the RNA editing enzyme is the adenosine deaminase acting on RNA (ADARs), such as hADARI and hADAR2 in humans or human cells.

Adenosine deaminases acting on RNA (ADARs) catalyze adenosine (A) to inosine (I) editing of RNA that possesses double-stranded (ds) structure. A-to-I RNA editing results in nucleotide substitution, because I is recognized as G instead of A both by ribosomes and by RNA polymerases. A-to-I substitution can also cause dsRNA destabilization, as I:U mismatch base pairs are less stable than A:U base pairs. A-to-I editing occurs with both viral and cellular RNAs, and affects a broad range of biological processes. These include virus growth and persistence, apoptosis and embryogenesis, neurotransmitter receptor and ion channel function, pancreatic cell function, and post-transcriptional gene regulation by microRNAs. Biochemical processes that provide a framework for understanding the physiologic changes following ADAR-catalyzed A-to-I (=G) editing events include mRNA translation by changing codons and hence the amino acid sequence of proteins; pre-mRNA splicing by altering splice site recognition sequences; RNA stability by changing sequences involved in nuclease recognition; genetic stability in the case of RNA virus genomes by changing sequences during viral RNA replication; and RNA-structure-dependent activities such as microRNA production or targeting or protein-RNA interactions.

Three human ADAR genes are known, of which two encode active deaminases (ADAR1 and ADAR2). Human ADAR3 (hADAR3) has been described in the prior art, but reportedly has no deaminase activity. Alternative promoters together with alternative splicing give rise to two protein size forms of ADAR1: an interferon-inducible ADAR1-p150 deaminase that binds dsRNA and Z-DNA, and a constitutively expressed ADAR1-p110 deaminase. ADAR2, like ADAR1-p110, is constitutively expressed and binds dsRNA. It is known that only the longer isoform of ADAR1 is capable of binding to the Z-DNA structure that can be comprised in the recruiting portion of the oligonucleotide construct according to the invention. Consequently, the level of the 150 kDa isoform present in the cell may be influenced by interferon, particularly interferon-gamma (IFN-gamma). hADARI is also inducible by TNF-alpha. This provides an opportunity to develop combination therapy, whereby interferon-gamma or TNF-alpha and oligonucleotide constructs comprising Z-DNA as recruiting portion according to the invention are administered to a patient either as a combination product, or as separate products, either simultaneously or subsequently, in any order. Certain disease conditions may already coincide with increased IFN-gamma or TNF-alpha levels in certain tissues of a patient, creating further opportunities to make editing more specific for diseased tissues.

Recruiting ADAR to specific sites of selected transcripts and deamination of adenosine regardless of neighboring bases holds great promise for the treatment of disease. In some embodiments, the oligonucleotide that is capable of effecting an adenosine deaminase acting on RNA (ADAR)-mediated adenosine to inosine alteration of the SNP comprises an ADAR-recruiting domain. The ADAR-recruiting domain portion may act to recruit an endogenous ADAR enzyme present in the cell. Such ADAR-recruiting domains do not require conjugated entities or presence of modified recombinant ADAR enzymes. Alternatively, the ADAR-recruiting portion may act to recruit a recombinant ADAR fusion protein that has been delivered to a cell or to a subject via an expression vector construct including a polynucleotide encoding an ADAR fusion protein. Such ADAR-fusion proteins may include the deaminase domain of ADAR1 or ADAR2 enzymes fused to another protein, e.g., to the MS2 bacteriophage coat protein.

In some embodiments, the ADAR is endogenously expressed in a cell. The cell is selected from the group consisting of a bacterial cell, a eukaryotic cell, a mammalian cell, and a human cell. In principle the invention can be used with cells from any mammalian species, but it is preferably used with a human cell.

The oligonucleotide capable of effecting an adenosine deaminase acting on RNA (ADAR)-mediated adenosine to inosine alteration of the SNP, e.g., an oligonucleotide as described herein, comprises a portion that has sequence complementarity to a target mRNA encoding the SNP associated with a disease. In some embodiments, a region of the oligonucleotide is complementary to target mRNA with the exception of at least one mismatch. The oligonucleotide includes a mismatch opposite the target adenosine. In some embodiments, the activity region of an oligonucleotide comprises the sequence complementary to the target mRNA. In some embodiments, this activity region is comprised in a complex comprising two oligonucleotides, wherein each oligonucleotide comprises an activity region and a duplex region.

The activity region of an oligonucleotide in a complex hybridizes to the target mRNA sequence, and the double-stranded region formed by the duplex regions of the two oligonucleotides in the complex can be recognized by ADAR, which facilitates the recruitment of ADAR to the target sequence. As a result, ADAR can catalyze the deamination reaction of the specific adenosine in the targeted mRNA into an inosine.

The methods of the present invention can be used with cells from any organ, e.g. skin, lung, heart, kidney, liver, pancreas, gut, muscle, gland, eye, brain, blood and the like. The invention is particularly suitable for modifying sequences in cells, tissues or organs implicated in a diseased state of a (human) subject.

The methods of the invention can also be used with mammalian cells which are not naturally present in an organism e.g. with a cell line or with an embryonic stem (ES) cell. The methods of the invention can be used with various types of stem cells, including pluripotent stem cells, totipotent stem cells, embryonic stem cells, induced pluripotent stem cells, etc.

The cells can be located in vitro or in vivo. One advantage of the invention is that it can be used with cells in situ in a living organism, but it can also be used with cells in culture. In some embodiments cells are treated ex vivo and are then introduced into a living organism (e.g. re-introduced into an organism from whom they were originally derived). In some embodiments, the cell is contacted in vivo. In other embodiments, the cell is ex vivo.

The methods of invention can also be used to edit target RNA sequences in cells within a so-called organoid. Organoids are self-organized three-dimensional tissue structures derived from stem cells. Such cultures can be crafted to replicate much of the complexity of an organ, or to express selected aspects of it like producing only certain types of cells (Lancaster & Knoblich, Science 2014, vol. 345 no. 6194 1247125). In a therapeutic setting they are useful because they can be derived in vitro from a patient's cells, and the organoids can then be re-introduced to the patient as autologous material which is less likely to be rejected than a normal transplant. Thus, according to another preferred embodiment, the invention may be practiced on organoids grown from tissue samples taken from a patient (e.g. from their gastrointestinal tract; see Sala et al. J Surg Res. 2009; 156(2):205-12, and Sato et al. Gastroenterology 201 1; 141: 1762-72). Upon RNA editing in accordance with the invention, the organoids, or stem cells residing within the organoids, may be used to transplant back into the patient to ameliorate organ function.

In some embodiments, the cells to be treated have a genetic mutation. The mutation may be heterozygous or homozygous. The invention can be used to modify point mutations, for example, to correct a G to A mutation. In other embodiments, the cells to be treated do not have a genetic mutation. The invention can be used to create point mutations, for example, to generate an A to G mutation.

Accordingly, the invention is not limited to correcting mutations, as it may instead be useful to change a wildtype sequence into a mutated sequence by applying oligonucleotides according to the invention. One example where it may be advantageous to modify a wildtype adenosine is to bring about skipping of an exon, for example by modifying an adenosine that happens to be a branch site required for splicing of said exon. Another example is where the adenosine defines or is part of a recognition sequence for protein binding, or is involved in secondary structure defining the stability of the mRNA. In some embodiments, however, the invention is used in the opposite way by introducing a disease-associated mutation into a cell line or an animal, in order to provide a useful research tool for the disease in question.

A mutation to be reverted through RNA editing may have arisen on the level of the chromosome or some other form of DNA, such as mitochondrial DNA, or RNA, including pre-mRNA, ribosomal RNA or mitochondrial RNA. A change to be made may be in a target RNA of a pathogen, including fungi, yeasts, parasites, kinetoplastids, bacteria, phages, viruses etc, with which the cell or subject has been infected. Subsequently, the editing may take place on the RNA level on a target sequence inside such cell, subject or pathogen. Certain pathogens, such as viruses, release their nucleic acid, DNA or RNA into the cell of the infected host (cell). Other pathogens reside or circulate in the infected host. The oligonucleotide constructs of the invention may be used to edit target RNA sequences residing in a cell of the infected eukaryotic host, or to edit a RNA sequence inside the cell of a pathogen residing or circulating in the eukaryotic host, as long as the cells where the editing is to take place contain an editing entity compatible with the oligonucleotide construct administered thereto.

Without intending to be bound by theory, the RNA editing through ADAR1 and ADAR2 is thought to take place on pre-mRNAs in the nucleus, during transcription or splicing. Editing of mitochondrial RNA codons or non-coding sequences in mature mRNAs is not excluded.

Deamination of an adenosine using the oligonucleotides disclosed herein includes any level of adenosine deamination, e.g., at least 1 deaminated adenosine within a target sequence (e.g., at least, 1, 2, 3, or more deaminated adenosines in a target sequence).

Adenosine deamination may be assessed by a decrease in an absolute or relative level of adenosines within a target sequence compared with a control level. The control level may be any type of control level that is utilized in the art, e.g., pre-dose baseline level, or a level determined from a similar subject, cell, or sample that is untreated or treated with a control (such as, e.g., buffer only control or inactive agent control).

Because the enzymatic activity of ADAR converts adenosines to inosines, adenosine deamination can alternatively be assessed by an increase in an absolute or relative level of inosines within a target sequence compared with a control level. Similarly, the control level may be any type of control level that is utilized in the art, e.g., pre-dose baseline level, or a level determined from a similar subject, cell, or sample that is untreated or treated with a control (such as, e.g., buffer only control or inactive agent control).

The levels of adenosines and/or inosines within a target sequence can be assessed using any of the methods known in the art for determining the nucleotide composition of a polynucleotide sequence. For example, the relative or absolute levels of adenosines or inosines within a target sequence can be assessed using nucleic acid sequencing technologies including but not limited to Sanger sequencing methods, Next Generation Sequencing (NGS; e.g., pyrosequencing, sequencing by reversible terminator chemistry, sequencing by ligation, and real-time sequencing) such as those offered on commercially available platforms (e.g., Illumina, Qiagen, Pacific Biosciences, Thermo Fisher, Roche, and Oxford Nanopore Technologies). Clonal amplification of target sequences for NGS may be performed using real-time polymerase chain reaction (also known as qPCR) on commercially available platforms from Applied Biosystems, Roche, Stratagene, Cepheid, Eppendorf, or Bio-Rad Laboratories. Additionally or alternatively, emulsion PCR methods can be used for amplification of target sequences using commercially available platforms such as Droplet Digital PCR by Bio-Rad Laboratories.

In certain embodiments, surrogate markers can be used to detect adenosine deamination within a target sequence. For example, effective treatment of a subject having a genetic disorder involving G-to-A mutations with an oligonucleotide of the present disclosure, as demonstrated by an acceptable diagnostic and monitoring criteria can be understood to demonstrate a clinically relevant adenosine deamination. In certain embodiments, the methods include a clinically relevant adenosine deamination, e.g., as demonstrated by a clinically relevant outcome after treatment of a subject with an oligonucleotide of the present disclosure.

Adenosine deamination in a gene of interest may be manifested by an increase or decrease in the levels of mRNA expressed by a first cell or group of cells (such cells may be present, for example, in a sample derived from a subject) in which a gene of interest is transcribed and which has or have been treated (e.g., by contacting the cell or cells with an oligonucleotide or complex of the present disclosure, or by administering an oligonucleotide or complex of the invention to a subject in which the cells are or were present) such that the expression of the gene of interest is increased or decreased, as compared to a second cell or group of cells substantially identical to the first cell or group of cells but which has not or have not been so treated (control cell(s) not treated with an oligonucleotide or complex or not treated with an oligonucleotide or complex targeted to the gene of interest). The degree of increase or decrease in the levels of mRNA of a gene of interest may be expressed in terms of:

$$\frac{( mRNA in control cells ) - ( mRNA in treated cells )}{( mRNA in control cells )} \times 100 \%$$

In other embodiments, change in the levels of a gene may be assessed in terms of a reduction of a parameter that is functionally linked to the expression of a gene of interest, e.g., protein expression of the gene of interest or signaling downstream of the protein. A change in the levels of the gene of interest may be determined in any cell expressing the gene of interest, either endogenous or heterologous from an expression construct, and by any assay known in the art.

A change in the level of expression of a gene of interest may be manifested by an increase or decrease in the level of the protein produced by the gene of interest that is expressed by a cell or group of cells (e.g., the level of protein expressed in a sample derived from a subject). As explained above, for the assessment of mRNA suppression, the change in the level of protein expression in a treated cell or group of cells may similarly be expressed as a percentage of the level of protein in a control cell or group of cells.

A control cell or group of cells that may be used to assess the change in the expression of a gene of interest includes a cell or group of cells that has not yet been contacted with an oligonucleotide of the present disclosure. For example, the control cell or group of cells may be derived from an individual subject (e.g., a human or animal subject) prior to treatment of the subject with an oligonucleotide.

The level of mRNA of a gene of interest that is expressed by a cell or group of cells may be determined using any method known in the art for assessing mRNA expression. In one embodiment, the level of expression of a gene of interest in a sample is determined by detecting a transcribed polynucleotide, or portion thereof, e.g., mRNA of the gene of interest. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating mRNA of the gene of interest may be detected using methods the described in PCT Publication WO2012/177906, the entire contents of which are hereby incorporated herein by reference. In some embodiments, the level of expression of the gene of interest is determined using a nucleic acid probe. The term “probe,” as used herein, refers to any molecule that is capable of selectively binding to a specific sequence, e.g. to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to the mRNA of a gene of interest. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of mRNA of a gene of interest.

An alternative method for determining the level of expression of a gene of interest in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of a gene of interest is determined by quantitative fluorogenic RT-PCR (i.e., the TAQMAN™ System) or the DUAL-GLO® Luciferase assay.

The expression levels of mRNA of a gene of interest may be monitored using a membrane blot (such as used in hybridization analysis such as northern, Southern, dot, and the like), or microwells, sample tubes, gels, beads or fibers (or any solid support including bound nucleic acids). See U.S. Pat. Nos. 5,770,722; 5,874,219; 5,744,305; 5,677,195; and 5,445,934, which are incorporated herein by reference. The determination of gene expression level may also include using nucleic acid probes in solution.

In some embodiments, the level of mRNA expression is assessed using branched DNA (bDNA) assays or real time PCR (qPCR). The use of this PCR method is described and exemplified in the Examples presented herein. Such methods can also be used for the detection of nucleic acids of the gene of interest.

The level of protein produced by the expression of a gene of interest may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of proteins produced by the gene of interest. Additionally, the above assays may be used to report a change in the mRNA sequence of interest that results in the recovery or change in protein function thereby providing a therapeutic effect and benefit to the subject, treating a disorder in a subject, and/or reducing of symptoms of a disorder in the subject.

### Methods of Treatment

The present invention also include methods of treating or preventing a disease or disorder. For example, the methods of the invention may be used to treat or prevent any diseases or disorders which may be caused by a guanosine to adenosine mutation, the introduction of a premature stop codon, or expression of an undesired protein. In some embodiments, the oligonucleotides or complexes for use in the methods of the invention, when introduced to a cell or a subject, can result in correction of a guanosine to adenosine mutation. In some embodiments, the oligonucleotides or complexes for use in the methods of the invention can result in turning off of a premature stop codon so that a desired protein is expressed. In some embodiments, the oligonucleotides or complexes for use in the methods of the invention can result in inhibition of expression of an undesired protein.

In some embodiments, a method of treating or preventing a disease or disorder includes restoring or modulating the function of a target protein. “Restoring function,” as used herein, refers to substitution of a mutated nucleotide (i.e., a SNP from a wildtype nucleotide) within a nucleotide sequence, wherein the substitution allows for expression of the wildtype protein or expression of a protein comprising a different amino acid compared to a wildtype protein, wherein the protein comprising a different amino acid has substantially similar function as wildtype. In some embodiments, restoring function comprises expression of a protein with an amino acid sequence conferring 50% or greater, 60% or greater, 70% or greater, 80% or greater, or 90% or greater activity as compared to the wildtype protein. The function of the wildtype protein may be any biological effect of this protein as measured in an in vitro or in vivo system. In some embodiments, restoring function comprises expression of a protein with an amino acid sequence conferring an increase in activity of 10% or greater, 20% or greater, 30% or greater, 40% or greater, 50% or greater, 60% or greater, 70% or greater, or 80% or greater as compared to the protein encoded by a nucleotide comprising the SNP.

An exemplary embodiment of restoring function would be nucleotide editing such that an adenosine to inosine alteration allows for substitution of a pathogenic amino acid with a wildtype amino acid or an amino acid that allows for normal function of the protein (i.e., a permissive amino acid). It is known in the art that proteins comprising a different amino acid sequence from wildtype may be tolerated and allow for normal function of the protein.

“Modulating function,” as used herein, refers to substitution of a mutated nucleotide (i.e., a SNP from a wildtype nucleotide) within a nucleotide sequence, wherein the substitution results in expression of a protein comprising a different amino acid compared to the wildtype protein, and wherein the protein expressing this amino acid is partially tolerated in one or more cellular process and allows for the greater function of the protein as compared to the function of the protein encoded by the nucleotide sequence comprising the SNP, but the function is less than that of the wildtype protein. In some embodiments, modulating function comprises expression of a protein with an amino acid sequence conferring 10% or greater, 20% or greater, 30% or greater, 40% or greater, 50% or greater, 60% or greater, 70% or greater, or 80% or greater activity as compared to the wildtype protein. In some embodiments, modulating function comprises expression of a protein with an amino acid sequence conferring an increase in activity of 10% or greater, 20% or greater, 30% or greater, 40% or greater, 50% or greater, 60% or greater, 70% or greater, or 80% or greater as compared to the protein encoded by a nucleotide comprising the SNP.

An example of modulating function would be an increase or decrease in the level of a normal or mutant protein, such as a suppressor transcription regulatory protein, as needed to ameliorate a disease. Such suppressor transcription regulatory proteins include NFkB or other regulatory factors.

In some embodiments, an adenosine is substituted for another nucleotide that replaces a pathogenic amino acid with an amino acid that confers the substantially similar protein activity as the wildtype amino acid. In some embodiments, an adenosine is substituted for another nucleotide that replaces the pathogenic amino acid with an amino acid that confers restored or modulated function as compared to a protein comprising the pathogenic amino acid.

In some embodiments, a complex of two oligonucleotides as described herein is prepared before administering to a subject. In some embodiments, the complex of two oligonucleotides is in solution prepared prior to the administering. In some embodiments, the complex is formed under physiological conditions, based on hybridizing of the duplex regions of two oligonucleotides to generate a double-stranded structure, wherein the duplex regions are substantially reverse complementary. In some embodiments, a solution comprising oligonucleotides is heated to reduce any intramolecular secondary structure formation (such as internal hairpins that may form, etc.), and the solution is then slowly cooled to allow for hybridizing of the duplex regions of two oligonucleotides and preparation of a complex.

In some embodiments, the subject is a human subject.

The methods of the invention thus may include a step of identifying a subject with a single nucleotide polymorphism (SNP) associated with a disease or disorder in an polynucleotide. Specifically, the methods of the invention include a step of identifying the presence of the desired nucleotide change or SNPs in the target RNA sequence, thereby verifying that the target RNA sequence has the disease causing mutations to be corrected or edited. This step will typically involve sequencing of the relevant part of the target RNA sequence, or a cDNA copy thereof (or a cDNA copy of a splicing product thereof, in case the target RNA is a pre-mRNA), and the sequence change can thus be easily verified. Alternatively the modifications may be assessed on the level of the protein (length, glycosylation, function or the like), or by some functional read-out.

The methods disclosed herein also include contacting the target polynucleotides with a single nucleotide polymorphism (SNP) associated with a disease or disorder in a cell or a subject (including a subject identified as being in need of such treatment, or a subject suspected of being at risk of disease and in need of such treatment) with an oligonucleotide or complex capable of effecting an adenosine deaminase acting on RNA (ADAR)-mediated adenosine to inosine alteration of the SNP associated with the disease or disorder, as described herein.

The oligonucleotides for use in the methods of the invention are designed to specifically target the mRNA of a subject (e.g., a human patient) in need thereof, and are capable of effecting an ADAR-mediated adenosine to inosine alteration in the SNPs associated with a disease or disorder. In some embodiments, the oligonucleotides or complexes are capable of recruiting the ADAR to the target mRNA, which then catalyze deamination of target adenosines in the target mRNA. Such treatment will be suitably introduced to a subject, particularly a human subject, suffering from, having, susceptible to, or at risk for developing the disease or disorder.

In one embodiment, the invention provides a method of monitoring treatment progress. The method includes the step of determining a level of diagnostic marker (Marker) (e.g., SNP associated with the disease or disorder) or diagnostic measurement (e.g., screen, assay) in a subject suffering from or susceptible to developing the disease or disorder, or symptoms associated with the disease or disorder in which the subject has been administered a therapeutic amount of a composition disclosed herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In some embodiments, cells are obtained from the subject and contacted with an oligonucleotide or complex of the invention as provided herein. In some embodiments, the cell is autologous, allogenic, or xenogenic to the subject. In some embodiments, cells removed from a subject and contacted ex vivo with an oligonucleotide or complex of the invention are re-introduced into the subject, optionally after the desired genomic modification has been effected or detected in the cells.

In some embodiments, the oligonucleotide or complex for use in the methods of the present disclosure is introduced to a subject such that the oligonucleotide or complex is delivered to a specific site within the subject. For example, in some embodiments the oligonucleotide or complex maybe intravitreally injected. The change in the expression of the gene of interest may be assessed using measurements of the level or change in the level of mRNA or protein produced by the gene of interest in a sample derived from a specific site within the subject.

In other embodiments, the oligonucleotide or complex is introduced into the cell or the subject in an amount and for a time effective to result in one (or more, e.g., two or more, three or more, four or more) of: (a) decrease in the proportion of mRNA that comprises adenosine at the target SNP within a target sequence of the gene of interest, (b) decrease the proportion of target protein comprising the pathogenic mutation, (c) delayed onset of a disease or disorder, (d) recovery or change in protein function or level of function, and (e) reduction in one or more of symptoms related to the disease or disorder.

Treating disorders associated with G-to-A mutations can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a compound or pharmaceutically acceptable salt of a compound described herein. A decrease in the mortality rate of a population may also be measured, for example, by calculating for a population the average number of disease-related deaths per unit time following completion of a first round of treatment with a compound or pharmaceutically acceptable salt of a compound described herein.

### A. Methods of Administration

The delivery of an oligonucleotide or complex for use in the methods of the invention to a cell e.g., a cell within a subject, such as a human subject (e.g., a subject in need thereof) can be achieved in a number of different ways. For example, delivery may be performed by contacting a cell with an oligonucleotide or complex of the invention either in vitro or in vivo. In vivo delivery may also be performed directly by administering a composition including an oligonucleotide or complex to a subject. Alternatively, in vivo delivery may be performed indirectly by administering one or more vectors that encode and direct the expression of the oligonucleotide. Exemplary vectors for in vivo delivery of an oligonucleotide or complex include lentiviral or adeno-associated virus (AAV) vectors. Combinations of in vitro and in vivo methods of contacting a cell are also possible. Contacting a cell may be direct or indirect. Furthermore, contacting a cell may be accomplished via a targeting ligand, including any ligand described herein or known in the art. In some embodiments, the targeting ligand is a carbohydrate moiety, e.g., a GalNAc₃ ligand, or any other ligand that directs the oligonucleotide to a site of interest.

Contacting of a cell with an oligonucleotide or complex may be done in vitro or in vivo. Known methods can be adapted for use with an oligonucleotide or complex of the invention (see e.g., Akhtar S. and Julian R L., (1992) Trends Cell. Biol. 2(5):139-144 and WO94/02595, which are incorporated herein by reference in their entireties). For in vivo delivery, factors to consider in order to deliver an oligonucleotide molecule include, for example, biological stability of the delivered molecule, prevention of non-specific effects, and accumulation of the delivered molecule in the target tissue. The non-specific effects of an oligonucleotide or complex can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering an oligonucleotide or complex systemically for the treatment of a disease, an oligonucleotide or complex can include alternative nucleobases, alternative sugar moieties, and/or alternative internucleoside linkages, or alternatively delivered using a drug delivery system; both methods act to prevent the rapid degradation of the oligonucleotide by endo- and exo-nucleases in vivo. Modification of the oligonucleotide or complex, or the pharmaceutical carrier thereof, can also permit targeting of the oligonucleotide or complex to the target tissue and avoid undesirable off-target effects. Oligonucleotide molecules can be modified by chemical conjugation to lipophilic groups such as cholesterol to enhance cellular uptake and prevent degradation. In an alternative embodiment, the oligonucleotide or complex can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of an oligonucleotide or complex (negatively charged) and also enhance interactions at the negatively charged cell membrane to permit efficient uptake of an oligonucleotide or complex by the cell. Cationic lipids, dendrimers, or polymers can either be bound to an oligonucleotide or complex, or induced to form a vesicle or micelle that encases an oligonucleotide or complex. The formation of vesicles or micelles further prevents degradation of the oligonucleotide or complex when administered systemically. In general, any methods of delivery of nucleic acids known in the art may be adaptable to the delivery of the oligonucleotides or complexes of the invention. Methods for making and administering cationic oligonucleotides or complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of oligonucleotides or complexes include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an oligonucleotide or a complex forms a further complex with cyclodextrin for systemic administration. Methods for administration and pharmaceutical compositions of oligonucleotides and cyclodextrins can be found in U.S. Pat. No. 7,427,605, which is herein incorporated by reference in its entirety. In some embodiments the oligonucleotides or complexes of the invention are delivered by polyplex or lipoplex nanoparticles. Methods for administration and pharmaceutical compositions of oligonucleotides (or complexes) and polyplex nanoparticles and lipoplex nanoparticles can be found in U.S. Patent Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

i. Membranous Molecular Assembly Delivery Methods

Oligonucleotides or complexes for use in the methods of the invention can also be delivered using a variety of membranous molecular assembly delivery methods including polymeric, biodegradable microparticle, or microcapsule delivery devices known in the art. For example, a colloidal dispersion system may be used for targeted delivery an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide or complex are delivered into the cell where the oligonucleotide or complex can specifically bind to a target RNA and can mediate ADAR-mediated RNA editing. In some cases, the liposomes are also specifically targeted, e.g., to direct the oligonucleotide or complex to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide or complex can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide or complex preparation is then added to the micelles that include the lipid component. The cationic groups on the lipid interact with the oligonucleotide or the complex and condense around the oligonucleotide to form a liposome. After condensation, the detergent is removed, e.g., by dialysis, to yield a liposomal preparation of an oligonucleotide or complex.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169. These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems including non-ionic surfactant and cholesterol. Non-ionic liposomal formulations including NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P. Pharma. Sci., 4(6):466).

Liposomes may also be sterically stabilized liposomes, including one or more specialized lipids that result in enhanced circulation lifetimes relative to liposomes lacking such specialized lipids. Examples of sterically stabilized liposomes are those in which part of the vesicle-forming lipid portion of the liposome (A) includes one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes including one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes including (1) sphingomyelin and (2) the ganglioside G_{M1} or a galactocerebroside sulfate ester. U.S. Pat. No. 5,543,152 (Webb et al.) discloses liposomes including sphingomyelin. Liposomes including 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides or complexes of two oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotides (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that include positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotides into the skin. In some implementations, liposomes are used for delivering oligonucleotides to epidermal cells and also to enhance the penetration of oligonucleotides into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems including non-ionic surfactant and cholesterol. Non-ionic liposomal formulations including Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with oligonucleotide are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application No. PCT/US2007/080331, filed Oct. 3, 2007 also describes formulations that are amenable to the present invention.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotide or complex for use in the methods of the invention can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

ii. Lipid Nanoparticle-Based Delivery Methods

Oligonucleotides or complexes of two oligonucleotides for use in the methods of in the invention may be fully encapsulated in a lipid formulation, e.g., a lipid nanoparticle (LNP), or other nucleic acid-lipid particles. LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to oligonucleotide ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

Non-limiting examples of cationic lipid include N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can include, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

### B. Combination Therapies

A method of the invention can be used alone or in combination with an additional therapeutic agent, e.g., other agents that treat the same disorder, or symptoms associated therewith, or in combination with other types of therapies to the disorder. In combination treatments, the dosages of one or more of the therapeutic compounds may be reduced from standard dosages when administered alone. For example, doses may be determined empirically from drug combinations and permutations or may be deduced by isobolographic analysis. Dosages of the compounds when combined should provide a therapeutic effect.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### III. Compositions for Use in the Methods of the Invention

The oligonucleotides or complexes for use in the methods of the invention may be utilized to deaminate target adenosines on a specific mRNA, e.g., an adenosine which may be deaminated to produce a therapeutic result, e.g., in a subject in need thereof.

Examples of modifications resulting from deamination of target adenosines within a target codon are provided in Table 1 below.

TABLE 1

| Target | Amino Acid Encoded by | Modified | Amino Acid Encoded |
|---|---|---|---|
| Codon | Target Codon | Codon | by Modified Codon |
| AAA | Lys | IAA | Glu |
| | | AIA | Arg |
| | | IIA | Gly |
| | | AII | Arg |
| | | IAI | Glu |
| | | III | Gly |
| AAC | Asn | IAC | Asp |
| | | AIC | Ser |
| | | IIC | Gly |
| AAG | Lys | IAG | Glu |
| | | AIG | Arg |
| | | IIG | Gly |
| AAU | Arg | IAU | Asp |
| | | AIU | Ser |
| | | IIU | Gly |
| ACA | Thr | ICA | Ala |
| | | ICI | Ala |
| ACC | Thr | ICC | Ala |
| ACG | Thr | ICG | Ala |
| ACU | Thr | ICU | Ala |
| AGA | Arg | IGA | Gly |
| | | IGI | Gly |
| AGC | Ser | IGC | Gly |
| AGG | Arg | IGG | Gly |
| AGU | Ser | IGU | Gly |
| AUA | Ile | IUA | Asp |
| | | AUI | Met |
| | | IUI | Val |
| AUC | Ile | IUC | Val |
| AUG | Met | IUG | Val |
| AUU | Ile | IUU | Val |
| CAA | Gln | CIA | Arg |
| | | CII | Arg |
| CAC | His | CIC | Arg |
| CAG | Gln | CIG | Arg |
| CAU | His | CIU | Arg |
| GAA | Glu | GIA | Gly |
| | | GII | Gly |
| GAC | Asp | GIC | Gly |
| GAG | Glu | GIG | Gly |
| GAU | Asp | GIU | Gly |
| UAA | Stop | UII | Trp |
| UGA | Stop | UGI | Trp |
| UAC | Tyr | UIC | Cys |
| UAG | Stop | UIG | Trp |
| UAU | Tyr | UIU | Cys |

Because the deamination of the adenosine to an inosine may result in a protein that is no longer suffering from the mutated A at the target position, the identification of the deamination into inosine may be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. When the presence of a target adenosine causes aberrant splicing, the read-out may be the assessment of whether the aberrant splicing is still taking place, or not, or less. On the other hand, when the deamination of a target adenosine is wanted to introduce a splice site, then similar approaches can be used to check whether the required type of splicing is indeed taking place. A very suitable manner to identify the presence of an inosine after deamination of the target adenosine is of course RT-PCR and sequencing, using methods that are well-known to the person skilled in the art.

In general, mutations in any target RNA that can be reversed using oligonucleotides or complexes according to the invention are G-to-A mutations, and oligonucleotides or complexes can be designed accordingly. Mutations that may be targeted using according to the invention also include C to A, U to A (T to A on the DNA level) in the case of recruiting adenosine deaminases. Although RNA editing in the latter circumstances may not necessarily revert the mutation to wildtype, the edited nucleotide may give rise to an improvement over the original mutation. For example, a mutation that causes an in frame stop codon—giving rise to a truncated protein, upon translation—may be changed into a codon coding for an amino acid that may not be the original amino acid in that position, but that gives rise to a (full length) protein with at least some functionality, at least more functionality than the truncated protein.

### Exemplary Targets

In various embodiments, the oligonucleotides provided herein are complementary to a target mRNA sequence comprising the SNP associated with a disease. Nonlimiting exemplary targets, along with the SNP associated with a disease, and target amino acid to be edited, are shown in Table 2.

TABLE 2
Exemplary target mRNAs and SNPs

| Target | Target | Reference | Accession No. |
|---|---|---|---|
| Protein | amino acid | SNP | for mRNA |
| LRRK2 | G2019S | rs34637584 | NM_198578.4 |
| LRRK2 | R1441H | rs34995376 | NM_198578.4 |
| ASS1 | G390R | rs121908641 | NM_000050.4 |
| ASS1 | E191K | rs777828000 | NM_000050.4 |
| OTOF | R1939Q | rs201326023 | NM_194248.3 |
| OTOF | Q829X | rs80356593 | NM_194248.3 |
| OTOF | R794H | rs80356592 | NM_194248.3 |
| ASL | V178M | rs28941473 | NM_001024943.2 |
| ASL | R193Q | rs373697663 | NM_001024943.2 |
| ASL | Splice variant | rs142637046 | NM_001024943.2 |
| ASL | E59K | rs869312985 | NM_001024943.2 |
| ASL | R12Q | rs145138923 | NM_001024943.2 |
| GJB2 | V37I | rs72474224 | NM_004004.6 |
| GJB2 | W24X | rs104894396 | NM_004004.6 |
| MCEP2 | R255X | rs61749721 | NM_004992 |
| MCEP2 | R168X | rs61748421 | NM_004992 |
| MCEP2 | R294X | rs61751362 | NM_004992 |
| MCEP2 | R270X | rs61750240 | NM_004992 |
| NRF2 | E79G | | |
| NRF2 | E82G | | |
| TCM1 | D572N | rs121908072 | NM_138691.3 |
| TCM1 | R389X | rs151001642 | NM_138691.3 |
| RS1 | E72K | rs104894928 | NM_000330.3 |
| RS1 | R102Q | rs61752068 | NM_000330.3 |
| ABCA4 | G1961E | rs1800553 | NM_000350.3 |
| SERPINA1 | E342K | rs28929474 | NM_000295.5 |

The sequence of a human NRF2 mRNA transcript can be found at National Center for Biotechnology Information (NCBI) RefSeq accession numbers NM_001145412.3, NM_001145413.3, NM_001313900.1, NM_001313901.1, NM_001313902.2, NM_001313903.1, NM_001313904.1 and NM_006164.5.

In some embodiments, a human SERPINA1 SNP is located at nucleotide position 1143 of Accession ID: NM_000295.5. In some embodiments, a mutant alpha-1 antitrypsin encoded by SERPINA1 comprises a E342K substitution of Protein Accession ID: NP_000286.3.

In some embodiments, a target amino acid (i.e., substitution) is at position 366 in the premature polypeptide, but upon cleavage of the first 24 amino acids in post-translational processing, the target position becomes amino acid 342 in the mature polypeptide.

In some embodiments, a cynomolgus monkey SERPINA1 SNP is located at nucleotide position 1095 of Accession ID:XM_005562106.2. In some embodiments, a mutant alpha-1 antitrypsin encoded by SERPINA1 comprises a K359E substitution of Protein Accession ID: XP_005562163.2. In some embodiments, the target amino acid position is 359 in the premature polypeptide sequence.

In some embodiments, a mouse Rab7 SNP is located at nucleotide position 1790 of Accession ID: NM_001293652.1. In some embodiments, the Protein Accession ID of mouse Ras-related protein Rab-7a encoded by Rab7 is NP_001280581.1. In some embodiments, the target nucleotide is comprised in an untranslated region of mRNA transcribed from Rab7.

In some embodiments, a human RAB7A SNP is located at nucleotide position 1589 of Accession ID: NM_004637.6. In some embodiments, the Protein Accession ID of human Ras-related protein Rab-7a encoded by RAB7A is NP_004628.4. In some embodiments, the target nucleotide is comprised in an untranslated region of mRNA transcribed from RAB7A.

### Oligonucleotide Agents or Complexes Thereof

In some embodiments, the duplex region of a first oligonucleotide (i.e., an oligonucleotide comprising a duplex region) hybridizes to the duplex region of a second oligonucleotide. In some embodiments, the first and second oligonucleotide have the same sequence, and the duplex region is reverse complementary to itself.

In some embodiments, oligonucleotides or complexes thereof may comprise duplex regions that reduce or avoid intramolecular formation of double-stranded segments (such as hairpins or stem-loop secondary structures). In some embodiments, the number and/or location of reverse complementary nucleotides within the duplex region bias towards formation of a duplex by the annealing of two oligonucleotide, in comparison to formation of a secondary structure within a single oligonucleotide. In a representative example, a first and a second identical oligonucleotides comprise duplex regions that can hybridize over the duplex regions via reverse complementary nucleotide sequence. In some embodiments, the melting temperature (Tm) of the double-stranded region formed by the annealing of the duplex region of the two identical oligonucleotides is substantially higher than the melting temperature of an intramolecular hairpin (i.e., stem-loop) formed by one of the oligonucleotides by itself.

In some embodiments, the Tm of an oligonucleotide is optimized (either via computer modeling or testing) to promote formation of a double-stranded region of two oligonucleotides annealed via their duplex regions versus formation of a hairpin within a single oligonucleotide.

In some embodiments, a solution comprising duplex oligonucleotides is cooled slowly to promote hybridization of the duplex regions of two oligonucleotides. In some embodiments, slower cooling promotes annealing of two oligonucleotides comprising duplex regions into a complex, as compared to formation of a hairpin within a single oligonucleotide.

In some embodiments, the activity region of one or both oligonucleotides comprised in a complex of two oligonucleotides are complementary to target mRNA sequence comprising the SNP associated with a disease. In some embodiments, the activity region of one or both oligonucleotides comprised in a complex of two oligonucleotides is complementary to target mRNA with the exception of at least one mismatch. In some embodiments, the activity region includes a mismatch opposite the target adenosine to be edited by ADAR.

The oligonucleotides or complexes are also capable of recruiting adenosine deaminase acting on RNA (ADAR) enzymes to deaminate selected adenosines on the target mRNA. In some embodiments, only one adenosine is deaminated. In some embodiments, 1, 2, or 3 adenosines are deaminated.

The oligonucleotides for use in the methods of the invention may further include modifications (e.g., alternative nucleotides) to increase stability and/or increase deamination efficiency.

Alternative nucleotides and nucleosides include those with modifications including, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. The nucleobase may also be an isonucleoside in which the nucleobase is moved from the C1 position of the sugar moiety to a different position (e.g. C2, C3, C4, or C5). Specific examples of oligonucleotide compounds or complexes of two oligonucleotides useful in the embodiments described herein include, but are not limited to alternative nucleosides containing modified backbones or no natural internucleoside linkages. Nucleotides and nucleosides having modified backbones include, among others, those that do not have a phosphorus atom in the backbone. For the purposes of this specification, and as sometimes referenced in the art, alternative RNAs that do not have a phosphorus atom in their internucleoside backbone can also be considered to be oligonucleosides. In some embodiments, an oligonucleotide will have a phosphorus atom in its internucleoside backbone.

In one embodiment, one or more of the nucleotides of the oligonucleotides of the invention, is naturally-occurring, and does not include, e.g., chemical modifications and/or conjugations known in the art and described herein. In another embodiment, one or more of the nucleotides of an oligonucleotide of the invention is chemically modified to enhance stability or other beneficial characteristics (e.g., alternative nucleotides). Without being bound by theory, it is believed that certain modification can increase nuclease resistance and/or serum stability, or decrease immunogenicity. For example, polynucleotides of the invention may contain nucleotides found to occur naturally in DNA or RNA (e.g., adenine, thymidine, guanosine, cytidine, uridine, or inosine) or may contain nucleotides which have one or more chemical modifications to one or more components of the nucleotide (e.g., the nucleobase, sugar, or phospho-linker moiety). Oligonucleotides er of the invention may be linked to one another through naturally-occurring phosphodiester bonds, or may be modified to be covalently linked through phosphorothiorate, 3′-methylenephosphonate, 5′-methylenephosphonate, 3′-phosphoamidate, 2′-5′ phosphodiester, guanidinium, S-methylthiourea, or peptide bonds.

Alternative internucleoside linkages include, for example, phosphorothioates, chiral phosphorothioates, phosphorodithioates, phosphoramidates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boronophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts, and free acid forms are also included.

In some embodiments, the at least one alternative internucleoside linkage is selected from the group consisting of a phosphorothioate internucleoside linkage, a 2′-alkoxy internucleoside linkage, and an alkyl phosphate internucleoside linkage. In some embodiments, the at least one alternative internucleoside linkage is at least one phosphorothioate internucleoside linkage.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Alternative internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable oligonucleotides include those in which both the sugar and the internucleoside linkage, i.e., the backbone, of the nucleotide units are replaced. The base units are maintained for hybridization with an appropriate nucleic acid target compound. One such oligomeric compound, a mimetic that has been shown to have excellent hybridization properties, is referred to as a peptide nucleic acid (PNA). In PNA compounds, the sugar of a nucleoside is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the oligonucleotides of the invention are described in, for example, in Nielsen et al., Science, 1991, 254, 1497-1500.

Some embodiments featured in the invention include oligonucleotides with phosphorothioate backbones and oligonucleotides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester backbone is represented as —O—P—O—CH₂-] of the above-referenced U.S. Pat. No. 5,489,677, and the amide backbones of the above-referenced U.S. Pat. No. 5,602,240. In some embodiments, the oligonucleotides featured herein have morpholino backbone structures of the above-referenced U.S. Pat. No. 5,034,506. In other embodiments, the oligonucleotides described herein include phosphorodiamidate morpholino oligomers (PMO), in which the deoxyribose moiety is replaced by a morpholine ring, and the charged phosphodiester inter-subunit linkage is replaced by an uncharged phophorodiamidate linkage, as described in Summerton, et al., Antisense Nucleic Acid Drug Dev. 1997, 7:63-70.

Alternative nucleosides and nucleotides can also contain one or more substituted sugar moieties. The oligonucleotides, e.g., oligonucleotides, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl (e.g., 2′-O—C₁-C₁₀ alkyl-nucleotide, a 2′-O—C₁-C₆ alkyl-nucleotide, 2′-O-methyl) or C2 to C₁₀ alkenyl and alkynyl. Exemplary suitable modifications include —O[(CH₂)ₙO]ₘCH₃, —O(CH₂)ₙOCH₃, —O(CH₂)ₙ—NH₂, —O(CH₂)ₙCH₃, —O(CH₂)ₙ—ONH₂, and —O(CH₂)ₙ—ON[(CH₂)·CH₃]₂, where n and m are from 1 to about 10. In other embodiments, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modification includes a 2′-methoxyethoxy (2′-O—CH₂CH₂OCH₃, also known as 2′-O-(2-methoxyethyl) or 2′-O-MOE) (Martin et al., Helv. Chin. Acta, 1995, 78:486-504) i.e., an alkoxy-alkoxy group. 2′-O-MOE nucleosides confer several beneficial properties to oligonucleotides including, but not limited to, increased nuclease resistance, improved pharmacokinetics properties, reduced non-specific protein binding, reduced toxicity, reduced immunostimulatory properties, and enhanced target affinity as compared to unmodified oligonucleotides.

Another exemplary alternative contains 2′-dimethylaminooxyethoxy, i.e., a —O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—(CH₂)₂—O—(CH₂)₂—N(CH₃)₂. Further exemplary alternatives include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other alternatives include 2′-methoxy (2′-OCH₃), 2′-aminopropoxy (2′-OCH₂CH₂CH₂NH₂) and 2′-fluoro (2′-F). Similar modifications can also be made at other positions on the nucleosides and nucleotides of an oligonucleotide, particularly the 3′ position of the sugar on the 3′ terminal nucleotide or in 2′-5′ linked oligonucleotides and the 5′ position of 5′ terminal nucleotide. Oligonucleotides can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920. The entire contents of each of the foregoing are hereby incorporated herein by reference.

In some embodiments, the at least one alternative sugar moiety is selected from the group consisting of a 2′-O-alkyl-sugar moiety, a 2′-O-methyl-sugar moiety, a 2′-amino-sugar moiety, a 2′-fluoro-sugar moiety, a 2′-O-MOE sugar moiety, an ANA sugar moiety deoxyribose sugar moiety, and a bicyclic nucleic acid. In some embodiments, the bicyclic sugar moiety is selected from an oxy-LNA sugar moiety, a thio-LNA sugar moiety, an amino-LNA sugar moiety, a cEt sugar moiety, and an ethylene-bridged (ENA) sugar moiety. In some embodiments, the ANA sugar moiety is a 2′-fluoro-ANA sugar moiety. In some embodiments, the at least one alternative sugar moiety is a 2′-O-methyl-sugar moiety, a 2′-fluoro-sugar moiety, or a 2′-O-MOE sugar moiety.

An oligonucleotide for use in the methods of the present invention can also include nucleobase (often referred to in the art simply as “base”) alternatives (e.g., modifications or substitutions). Unmodified or natural nucleobases include the purine bases adenine (A) and guanine (G), and the pyrimidine bases thymine (T), cytosine (C) and uracil (U). Alternative nucleobases include other synthetic and natural nucleobases such as 5-methylcytosine, 5-hydroxymethylcytosine, 5-formylcytosine, 5-carboxycytosine, pyrrolocytosine, dideoxycytosine, uracil, 5-methoxyuracil, 5-hydroxydeoxyuracil, dihydrouracil, 4-thiouracil, pseudouracil, 1-methyl-pseudouracil, deoxyuracil, 5-hydroxybutynl-2′-deoxyuracil, xanthine, hypoxanthine, 7-deaza-xanthine, thienoguanine, 8-aza-7-deazaguanine, 7-methylguanine, 7-deazaguanine, 6-aminomethyl-7-deazaguanine, 8-aminoguanine, 2,2,7-trimethylguanine, 8-methyladenine, 8-azidoadenine, 7-methyladenine, 7-deazaadenine, 3-deazaadenine, 2,6-diaminopurine, 2-aminopurine, 7-deaza-8-aza-adenine, 8-amino-adenine, thymine, dideoxythymine, 5-nitroindole, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 8-azaguanine and 8-azaadenine, and 3-deazaguanine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, Antisense Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines, and N-2, N-6 and 0-6 substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., Antisense Research and Applications, CRC Press, Boca Raton, 1993, pp. 276-278) and are exemplary base substitutions, even more particularly when combined with 2′-O-methoxyethyl sugar modifications.

In some embodiments, the at least one alternative nucleobase is selected from the group consisting of 5-methylcytosine, 5-hydroxycytosine, 5-methoxycytosine, N4-methylcytosine, N3-Methylcytosine, N4-ethylcytosine, pseudoisocytosine, 5-fluorocytosine, 5-bromocytosine, 5-iodocytosine, 5-aminocytosine, 5-ethynylcytosine, 5-propynylcytosine, pyrrolocytosine, 5-aminomethylcytosine, 5-hydroxymethylcytosine, naphthyridine, 5-methoxyuracil, pseudouracil, dihydrouracil, 2-thiouracil, 4-thiouracil, 2-thiothymine, 4-thiothymine, 5,6-dihydrothymine, 5-halouracil, 5-propynyluracil, 5-aminomethyluracil, 5-hydroxymethyluracil, hypoxanthine, 7-deazaguanine, 8-aza-7-deazaguanine, 7-aza-2,6-diaminopurine, thienoguanine, N1-methylguanine, N2-methylguanine, 6-thioguanine, 8-methoxyguanine, 8-allyloxyguanine, 7-aminomethyl-7-deazaguanine, 7-methylguanine, imidazopyridopyrimidine, 7-deazaadenine, 3-deazaadenine, 8-aza-7-deazaadenine, 8-aza-7-deazaadenine, N1-methyladenine, 2-methyladenine, N6-methyladenine, 7-methyladenine, 8-methyladenine, or 8-azidoadenine.

In some embodiments, the at least one alternative nucleobase is selected from the group consisting of 2-amino-purine, 2,6-diamino-purine, 3-deaza-adenine, 7-deaza-adenine, 7-methyl-adenine, 8-azido-adenine, 8-methyl-adenine, 5-hydroxymethyl-cytosine, 5-methyl-cytosine, pyrrolo-cytosine, 7-aminomethyl-7-deaza-guanine, 7-deaza-guanine, 7-methyl-guanine, 8-aza-7-deaza-guanine, thieno-guanine, hypoxanthine, 4-thio-uracil, 5-methoxy-uracil, dihydro-uracil, or pseudouracil.

Representative U.S. patents that teach the preparation of certain of the above noted alternative nucleobases as well as other alternative nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In other embodiments, the sugar moiety in the nucleotide may be a ribose molecule, optionally having a 2′-O-methyl, 2′-O-MOE, 2′-F, 2′-amino, 2′-O-propyl, 2′-aminopropyl, or 2′-OH modification.

An oligonucleotide for use in the methods of the present invention can include one or more bicyclic sugar moieties. A “bicyclic sugar” is a furanosyl ring modified by the bridging of two atoms. A “bicyclic nucleoside” (“BNA”) is a nucleoside having a sugar moiety including a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include one or more locked nucleosides. A locked nucleoside is a nucleoside having a modified ribose moiety in which the ribose moiety includes an extra bridge connecting the 2′ and 4′ carbons. In other words, a locked nucleoside is a nucleoside including a bicyclic sugar moiety including a 4′-CH₂—O-2′ bridge. This structure effectively “locks” the ribose in the 3′-endo structural conformation. The addition of locked nucleosides to oligonucleotides has been shown to increase oligonucleotide stability in serum, and to reduce off-target effects (Grunweller, A. et al., (2003) Nucleic Acids Research 31(12):3185-3193). Examples of bicyclic nucleosides for use in the polynucleotides of the invention include without limitation nucleosides including a bridge between the 4′ and the 2′ ribosyl ring atoms. In certain embodiments, the polynucleotide agents of the invention include one or more bicyclic nucleosides including a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and j-D-ribofuranose (see WO 99/14226).

An oligonucleotide for use in the methods of the invention can also be modified to include one or more constrained ethyl nucleotides. As used herein, a “constrained ethyl nucleotide” or “cEt” is a locked nucleic acid including a bicyclic sugar moiety including a 4′-CH(CH3)-O-2′ bridge. In one embodiment, a constrained ethyl nucleotide is in the S conformation referred to herein as “S-cEt.”

An oligonucleotide for use in the methods of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an oligonucleotide for use in the methods of the invention includes one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule may also be modified with a cyclopropane ring to produce a tricyclodeoxynucleic acid (tricyclo DNA). The ribose moiety may be substituted for another sugar such as 1,5,-anhydrohexitol, threose to produce a threose nucleoside (TNA), or arabinose to produce an arabino nucleoside. The ribose molecule can also be replaced with non-sugars such as cyclohexene to produce cyclohexene nucleoside or glycol to produce glycol nucleosides.

The ribose molecule can also be replaced with non-sugars such as cyclohexene to produce cyclohexene nucleic acid (CeNA) or glycol to produce glycol nucleic acids (GNA). Potentially stabilizing modifications to the ends of nucleotide molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other alternatives chemistries of an oligonucleotide of the invention include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Exemplary oligonucleotides for use in the methods of the invention include sugar-modified nucleosides and may also include DNA or RNA nucleosides. In some embodiments, the oligonucleotide includes sugar-modified nucleosides and DNA nucleosides. Incorporation of alternative nucleosides into the oligonucleotide of the invention may enhance the affinity of the oligonucleotide for the target nucleic acid. In that case, the alternative nucleosides can be referred to as affinity enhancing alternative nucleotides.

In some embodiments, the oligonucleotide includes at least 1 alternative nucleoside, such as at least 2, at least 3, at least 4, at least 5, at least 6, at least 7, at least 8, at least 9, at least 10, at least 11, at least 12, at least 13, at least 14, at least 15 or at least 16 alternative nucleosides. In other embodiments, the oligonucleotides include from 1 to 10 alternative nucleosides, such as from 2 to 9 alternative nucleosides, such as from 3 to 8 alternative nucleosides, such as from 4 to 7 alternative nucleosides, such as 6 or 7 alternative nucleosides. In an embodiment, the oligonucleotide of the invention may include alternatives, which are independently selected from these three types of alternative (alternative sugar moiety, alternative nucleobase, and alternative internucleoside linkage), or a combination thereof. Preferably the oligonucleotide includes one or more nucleosides including alternative sugar moieties, e.g., 2′ sugar alternative nucleosides. In some embodiments, the oligonucleotide of the invention include the one or more 2′ sugar alternative nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, ANA, 2′-fluoro-ANA, and BNA (e.g., LNA) nucleosides. In some embodiments, the one or more alternative nucleoside is a BNA.

In some embodiments, at least 1 of the alternative nucleosides is a BNA (e.g., an LNA), such as at least 2, such as at least 3, at least 4, at least 5, at least 6, at least 7, or at least 8 of the alternative nucleosides are BNAs. In a still further embodiment, all the alternative nucleosides are BNAs.

In a further embodiment the oligonucleotide includes at least one alternative internucleoside linkage. In some embodiments, the internucleoside linkages within the contiguous nucleotide sequence are phosphorothioate or boronophosphate internucleoside linkages. In some embodiments, all the internucleotide linkages in the contiguous sequence of the oligonucleotide are phosphorothioate linkages. In some embodiments the phosphorothioate linkages are stereochemically pure phosphorothioate linkages. In some embodiments, the phosphorothioate linkages are Sp phosphorothioate linkages. In other embodiments, the phosphorothioate linkages are Rp phosphorothioate linkages.

In some embodiments, the oligonucleotide for use in the methods of the invention includes at least one alternative nucleoside which is a 2′-O-MOE-RNA, such as 2, 3, 4, 5, 6, 7, 8, 9, or 10 2′-O-MOE-RNA nucleoside units. In some embodiments, the 2′-O-MOE-RNA nucleoside units are connected by phosphorothioate linkages. In some embodiments, at least one of said alternative nucleoside is 2′-fluoro DNA, such as 2, 3, 4, 5, 6, 7, 8, 9, or 10 2′-fluoro-DNA nucleoside units. In some embodiments, the oligonucleotide of the invention includes at least one BNA unit and at least one 2′ substituted alternative nucleoside. In some embodiments of the invention, the oligonucleotide includes both 2′ sugar modified nucleosides and DNA units.

In certain embodiments of the invention, substantially all of the nucleotides of an oligonucleotide of the invention are alternative nucleotides. In other embodiments of the invention, all of the nucleotides of an oligonucleotide of the invention are alternative nucleotides. Oligonucleotides of the invention in which “substantially all of the nucleotides are alternative nucleotides” are largely but not wholly modified and can include no more than 5, 4, 3, 2, or 1 naturally-occurring nucleotides. In still other embodiments of the invention, an oligonucleotide of the invention can include no more than 5, 4, 3, 2, or 1 alternative nucleotides.

In some embodiments, the oligonucleotide of the invention may further include a 5′ cap structure. In some embodiments, the 5′ cap structure is a 2,2,7-trimethylguanosine cap.

The oligonucleotides of the invention can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. For example, an oligonucleotide of the invention can be synthesized by standard methods known in the art as further discussed below, e.g., by use of an automated DNA synthesizer, such as are commercially available from, for example, Biosearch, Applied Biosystems, Inc.

The oligonucleotide compound can be prepared using solution-phase or solid-phase organic synthesis or both. Organic synthesis offers the advantage that the oligonucleotide including unnatural or alternative nucleotides can be easily prepared. Single-stranded oligonucleotides of the invention can be prepared using solution-phase or solid-phase organic synthesis or both.

### A. Exemplary Oligonucleotides

Oligonucleotides capable of effecting an adenosine deaminase acting on RNA (ADAR)-mediated adenosine to inosine alteration of an adenosine in a target mRNA are provided herein. In some embodiments, these oligonucleotides are comprised in a complex of two oligonucleotides. In various embodiment, one or more of the nucleotides of the oligonucleotide of the invention, is naturally-occurring, and does not include, e.g., chemical modifications and/or conjugations known in the art and described herein. In some embodiment, one or more of the nucleotides of an oligonucleotide of the invention is chemically modified to enhance stability or other beneficial characteristics (for example, alternative nucleotides). Without being bound by theory, it is believed that certain modification can increase nuclease resistance and/or serum stability, or decrease immunogenicity. For example, oligonucleotides of the invention may contain nucleotides found to occur naturally in DNA or RNA (e.g., adenine, thymidine, guanosine, cytidine, uridine, or inosine) or may contain nucleotides which have one or more chemical modifications to one or more components of the nucleotide (e.g., the nucleobase, sugar, or phospho-linker moiety). Oligonucleotides of the invention may be linked to one another through naturally-occurring phosphodiester bonds, or may be modified to be covalently linked through phosphorothiorate, 3′-methylenephosphonate, 5′-methylenephosphonate, 3′-phosphoamidate, 2′-5′ phosphodiester, guanidinium, S-methylthiourea, or peptide bonds.

In certain embodiments of the invention, substantially all of the nucleotides of an oligonucleotide of the invention are alternative nucleotides. Oligonucleotides of the invention in which “substantially all of the nucleotides are alternative nucleotides” are largely but not wholly modified and can include no more than 10, 9, 8, 7, 6, 5, 4, 3, 2, or 1 naturally-occurring nucleotides.

In some embodiments, an oligonucleotide comprises an activity region and a duplex region.

In some embodiments, an oligonucleotide includes the activity region structure:

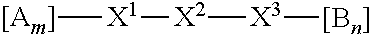

wherein each of A and B is a nucleotide; m and n are each, independently, an integer from 2 to 50; X¹, X², and X³ are each, independently, a nucleotide.

In some embodiments, an oligonucleotide of the present invention comprises a duplex region comprising 16-26 nucleotides. In some embodiments, a 5′ portion and a 3′ portion of the duplex region are substantially reverse complementary to one another. In some embodiments, the sequence of modifications in the duplex region may reduce formation of a hairpin.

In some embodiments, the duplex region comprises 15-30, 14-29, 13-28, 12-27, 11-26, 10-25, 9-24, 8-23, 12-28, 13-27, 14-26, 15-25, 16-24, 17-23, 18-22, or 19-21 nucleotides, or 19-20 nucleotides. In some embodiments, the duplex region comprises 14, 15, 16, 17, 18, 19, 20, 21, 22, 23, 24, or 25 nucleotides. In some embodiments, the duplex region comprises 18-22 nucleotides.

In some embodiments, activity region comprises 15-30, 14-29, 13-28, 12-27, 11-26, 10-25, 9-24, 8-23, 12-28, 13-27, 14-26, 15-25, 16-24, 17-23, 18-22, or 19-21 nucleotides. In some embodiments, the activity region comprises 14, 15, 16, 17, 18, 19, 20, 21, 22, 23, 24, or 25 nucleotides. In some embodiments, the activity region comprises 15-22 nucleotides. An exemplary activity region may comprise an 18mer sequence comprising 6 nucleotides 5′ of a triplet, 3 nucleotides of a triplet, and 9 nucleotides 3′ of the triplet.

In some embodiments, the triplet comprises nucleotides termed X¹-X²-X³. X¹-X²-X3 are sequential nucleotides without any other intervening nucleotides. X¹ is the 5′ nucleotide in the triplet, X² is the central nucleotide in the triplet, and X³ is the 3′ nucleotide in the triplet. X² is opposite the adenosine to be edited in the target mRNA when the oligonucleotide is hybridized to the target mRNA.

In some embodiments, the triplet is located internally within the oligonucleotide, meaning that none of the nucleotides comprised in the triplet are terminal nucleotides (i.e., the triplet is flanked by [Aₘ] and/or [Bₙ] nucleotides), wherein [Aₘ] is 5′ of the triplet and [Bₙ] is 3′ of the triplet. In some embodiments, the [Aₘ] and/or [Bₙ] nucleotides comprise two or more nucleotides. In some embodiments, the [Aₘ] and/or [Bₙ] nucleotides comprise four or more nucleotides. In some embodiments, the [Aₘ] nucleotides comprise two or more nucleotides and the [Bₙ] nucleotides comprise four or more nucleotides.

In some embodiments, the duplex regions of two oligonucleotides form a double-stranded region at physiological conditions. Accordingly, this double-stranded region can be formed or substantially maintained after administration to a subject. In some embodiments, this double-stranded region is present when two oligonucleotides (hybridized together by their duplex regions) reach a target cell.

In some embodiments, this double-stranded region formed by the duplex regions of two oligonucleotides comprises an ADAR recruiting domain. While in other oligonucleotides, a hairpin structure can form an ADAR recruiting domain, the present constructs form a double-stranded ADAR-recruiting domain from the duplex regions of two separate oligonucleotides. In some embodiments, forming an ADAR recruiting domain by hybridization of the duplex regions of two oligonucleotides allows for manufacture of significantly shorter oligonucleotides without full hairpin secondary structures.

In some embodiments, the duplex region is 5′ of the activity region. In some embodiments, the duplex region is 3′ of the activity region.

In some embodiments, the double-stranded region formed by the duplex region of two oligonucleotides is 15-30, 14-29, 13-28, 12-27, 11-26, 10-25, 9-24, 8-23, 12-28, 13-27, 14-26, 15-25, 16-24, 17-23, 18-22, or 19-21 base pairs (including mismatches) long. In some embodiments, the duplex region consists of 11-26 or 18-22 nucleotides.

In some embodiments, the double-stranded region formed by the duplex region of two oligonucleotides comprises 0, 1, 2, 3, 4, or 5 mismatches.

In some embodiments, the oligonucleotide consists of 20-80 nucleotides, or 20-70 nucleotides, or 20-60 nucleotides, or 30-60 nucleotides, or 30-50 nucleotides.

In some embodiments, at least one nucleotide of A is a 2′-F-nucleotide, wherein at least one 2′-F-nucleotide is at a position selected from +8, +3, −3, −7, −19 and −22, wherein X² is position 0 and X¹ is position −1.

In some embodiments, the oligonucleotide comprises 1, 2, 3, or 4 2′-F-nucleotides. That is, in some embodiments, the oligonucleotide does not comprise any 2′-F-nucleotides other than 2′-F-nucleotides at one or more of those positions.

In some embodiments, all purines comprised in the duplex region of the oligonucleotide are 2′-fluoro nucleotides, and all pyrimidines comprised in the duplex region of oligonucleotide are 2′-O-methoxyethyl-nucleotides.

In some embodiments, at least one nucleotide of [Aₘ] and/or at least one nucleotide of [Bₙ] is a nuclease-resistant nucleotide. In some embodiments, at least one nucleotide of [Aₘ] and/or at least one nucleotide of [Bₙ] is an alternative nucleotide. In some embodiments, [Bₙ] does not comprise any 2′-F-nucleotides. In some embodiments, each nucleotide of [Aₘ] that is not a 2′-F-nucleotide is a nuclease-resistant nucleotide. In some embodiments, each nucleotide of [Bₙ] is a nuclease-resistant nucleotide. In some embodiments, each nucleotide of [Aₘ] that is not a 2′-F-nucleotide is an alternative nucleotide. In some embodiments, each nucleotide of [Bₙ] is an alternative nucleotide.

In some embodiments, at least one nucleotide of [Aₘ] and/or at least one nucleotide of [Bₙ] is selected from a 2′-O—C₁-C₆ alkyl-nucleotide, a 2′-amino-nucleotide, an arabino nucleic acid-nucleotide, a bicyclic-nucleotide, a 2′-F-nucleotide, a 2′-O-methoxyethyl-nucleotide, a constrained ethyl (cEt)-nucleotide, a LNA-nucleotide, a ribonucleotide, and a DNA-nucleotide. In some embodiments, at least one nucleotide of [Aₘ] and/or at least one nucleotide of [Bₙ] is selected from a 2′-O-methyl-nucleotide, a 2′-F-nucleotide, a 2′-O-methoxyethyl-nucleotide, a cEt-nucleotide, a LNA-nucleotide, a ribonucleotide, and a DNA-nucleotide.

In some embodiments, each nucleotide of [Aₘ] that is not a 2′-F-nucleotide is independently selected from a 2′-O—C₁-C₆ alkyl-nucleotide, a 2′-amino-nucleotide, an arabino nucleic acid-nucleotide, a bicyclic-nucleotide, a 2′-O-methoxyethyl-nucleotide, a constrained ethyl (cEt)-nucleotide, a LNA-nucleotide, a ribonucleotide and a DNA-nucleotide. In some embodiments, each nucleotide of [Aₘ] that is not a 2′-F-nucleotide is independently selected from a 2′-O-methyl-nucleotide, a 2′-O-methoxyethyl-nucleotide, a cEt-nucleotide, a LNA-nucleotide, a ribonucleotide, and a DNA-nucleotide. In some embodiments, each nucleotide of [Aₘ] that is not a 2′-F-nucleotide is a 2′-O-methyl-nucleotide.

In some embodiments, each nucleotide of [Bₙ] is independently selected from a 2′-O—C₁-C₆ alkyl-nucleotide, a 2′-amino-nucleotide, an arabino nucleic acid-nucleotide, a bicyclic-nucleotide, a 2′-F-nucleotide, a 2′-O-methoxyethyl-nucleotide, a constrained ethyl (cEt)-nucleotide, a LNA-nucleotide, a ribonucleotide, and a DNA-nucleotide. In some embodiments, each nucleotide of [Bₙ] is independently selected from a 2′-O-methyl-nucleotide, a 2′-F-nucleotide, a 2′-O-methoxyethyl-nucleotide, a cEt-nucleotide, a LNA-nucleotide, a ribonucleotide, and a DNA-nucleotide. In some embodiments, each nucleotide of [Bₙ] is a 2′-O-methyl-nucleotide.

In various embodiments, at least one internucleoside linkage of the oligonucleotide is a phosphorothioate internucleoside linkage. In some embodiments, at least one phosphorothioate internucleoside linkage is stereopure. In some embodiments, the oligonucleotide comprises at least four phosphorothioate internucleoside linkages at one or both of the 5′ and 3′ termini of the oligonucleotide.

In some embodiments, X¹, X², and X³ are each independently selected from a 2′-O-C₁-C₆ alkyl-nucleotide, a 2′-amino-nucleotide, an arabino nucleic acid-nucleotide, a bicyclic-nucleotide, a 2′-F-nucleotide, a 2′-O-methoxyethyl-nucleotide, a constrained ethyl (cEt)-nucleotide, a LNA-nucleotide, and a DNA-nucleotide. In some embodiments, X¹, X², and X³ are each independently selected from 2′-O-methyl-nucleotide, a 2′-F-nucleotide, a 2′-O-methoxyethyl-nucleotide, a cEt-nucleotide, a LNA-nucleotide, and a DNA-nucleotide. In some embodiments, X² is not a 2′-O-methyl-nucleotide. In some embodiments, X² is a 2′-deoxyribonucleotide. In some such embodiments, and X¹ and/or X³ are alternative nucleotides. In some embodiments, X¹, X², and X³ are 2′-deoxyribonucleotides. In some embodiments, X² comprises a cytosine or 5′-methyl cytosine nucleobase. In some such embodiments, when the oligonucleotide is hybridized to a target mRNA, X² forms a mismatch with an adenosine in the target mRNA.

In some embodiments, [Aₘ] is 5-80, 5-70, 5-60, 5-50, 5-40, 5-30, 5-20, 5-10, 10-80, 10-70, 10-60, 10-50, 10-40, 10-30, or 10-20 nucleotides. In some embodiments, [Bₙ] is 5-80, 5-70, 5-60, 5-50, 5-40, 5-30, 5-20, 5-10, 10-80, 10-70, 10-60, 10-50, 10-40, 10-30, or 10-20 nucleotides.

The activity region of an oligonucleotide may be at least 70%, at least 75%, at least 80%, at least 85%, at least 90%, or at least 95% complementary to a target mRNA. In some embodiments, the activity region of the oligonucleotide is complementary to a target mRNA comprising a single nucleotide polymorphism (SNP) associated with a disease or disorder. In some embodiments, the target mRNA encodes a protein comprising a pathogenic amino acid resulting from the SNP.

Further, it is contemplated that for any sequence identified herein, further optimization could be achieved by systematically either adding or removing linked nucleosides to generate longer or shorter sequences. Further still, such optimized sequences can be adjusted by, e.g., the introduction of alternative nucleosides, alternative sugar moieties, and/or alternative internucleosidic linkages as described herein or as known in the art, including alternative nucleosides, alternative sugar moieties, and/or alternative internucleosidic linkages as known in the art and/or discussed herein to further optimize the molecule (e.g., increasing serum stability or circulating half-life, increasing thermal stability, enhancing transmembrane delivery, targeting to a particular location or cell type, and/or increasing interaction with RNA editing enzymes (e.g., ADAR)).

In some embodiments, an ADAR fusion protein is administered to the cell or to the subject using an expression vector construct including a polynucleotide encoding an ADAR fusion protein. In some embodiments, the ADAR fusion protein includes a deaminase domain of ADAR fused to an MS2 bacteriophage coat protein. In some embodiments, the deaminase domain of ADAR is a deaminase domain of ADAR1. In some embodiments, the deaminase domain of ADAR is a deaminase domain of ADAR2. The ADAR fusion protein may be a fusion protein described in Katrekar et al. Nature Methods, 16(3): 239-42 (2019), the ADAR fusion protein of which is herein incorporated by reference.

In some embodiments, a complex comprises two oligonucleotides described herein. In some embodiments, the complex comprises two oligonucleotides, wherein substantially all of the duplex regions of the two oligonucleotides are hybridized to each other, with 0, 1, 2, 3, 4, or 5 mismatches.

In some embodiments, the two oligonucleotides comprised in a complex are the same. In some embodiments, the complex comprises a double-stranded region formed by the duplex regions of the two oligonucleotides.

In some embodiments, a complex of two oligonucleotides further comprises target mRNA hybridized to the activity region of one or both oligonucleotides in the complex.

### B. Oligonucleotide Conjugated to Ligands

Oligonucleotides provided herein may be chemically linked to one or more ligands, moieties, or conjugates that enhance the activity, cellular distribution, or cellular uptake of the oligonucleotide. In some embodiments, one or both both oligonucleotides comprised in a complex are chemically linked to one or more ligands, moieties, or conjugates. In some embodiments, one oligonucleotide comprised in a complex of two oligonucleotides is chemically linked to one or more ligands, moieties, or conjugates, while the other oligonucleotide is not.

Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In one embodiment, a ligand alters the distribution, targeting, or lifetime of an oligonucleotide agent into which it is incorporated. In some embodiments, a ligand provides an enhanced affinity for a selected target, e.g., molecule, cell or cell type, compartment, e.g., a cellular or organ compartment, tissue, organ, or region of the body, as, e.g., compared to a species absent such a ligand.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic ionizable lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the oligonucleotide agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an oligonucleotide as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that include a number of phosphorothioate linkages are also known to bind to serum protein, thus short oligonucleotides, e.g., oligonucleotides of about 5 bases, 10 bases, 15 bases, or 20 bases, including multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated oligonucleotides of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides of the present invention, such as the ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

i. Lipid Conjugates

In one embodiment, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid-based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In another aspect, the ligand is a moiety, e.g., a vitamin, which is taken up by a target cell, e.g., a proliferating cell. Exemplary vitamins include vitamin A, E, and K.

ii. Cell Permeation Agents

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ; SEQ ID NO. 45) and the Drosophila Antennapedia protein (RQIKIWFQNRRMKWKK; SEQ ID NO. 46) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to an oligonucleotide agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidomimetics may include D-amino acids, as well as synthetic RGD mimics. In addition to RGD, one can use other moieties that target the integrin ligand. Some conjugates of this ligand target PECAM-1 or VEGF.

A cell permeation peptide is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin, or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

iii. Carbohydrate Conjugates

In some embodiments of the compositions and methods of the invention, an oligonucleotide further includes a carbohydrate. The carbohydrate conjugated oligonucleotide is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8). Representative carbohydrates include dextran, pullulan, chitin, chitosan, inulin, cyclodextrin, N-acetylglucosamine (GlcNAc), N-acetylgalactosamine (GalNAc), or hyaluronic acid.

In one embodiment, a carbohydrate conjugate for use in the compositions and methods of the invention is a monosaccharide.

In some embodiments, the carbohydrate conjugate further includes one or more additional ligands as described above, such as, but not limited to, a PK modulator and/or a cell permeation peptide.

Additional carbohydrate conjugates (and linkers) suitable for use in the present invention include those described in PCT Publication Nos. WO 2009073809, WO 2014/179620, and WO 2014/179627, the entire contents of each of which are incorporated herein by reference.

iv. Linkers

In some embodiments, the conjugate or ligand described herein can be attached to an oligonucleotide with various linkers that can be cleavable or non-cleavable.

Linkers typically include a direct bond or an atom such as oxygen or sulfur, a unit such as NR⁸, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times, or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissues. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

a. Redox Cleavable Linking Groups

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular oligonucleotide moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one embodiment, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

b. Phosphate-Based Cleavable Linking Groups

In another embodiment, a cleavable linker includes a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(OR^{k})—O—, —O—P(S)(OR^{k})—O—, —O—P(S)(SR^{k})—O—, —S—P(O)(OR^{k})—O—, —O—P(O)(OR^{k})—S—, —S—P(O)(OR^{k})—S—, —O—P(S)(OR^{k})—S—, —S—P(S)(OR^{k})—O—, —O—P(O)(R^{k})—O—, —O—P(S)(R^{k})—O—, —S—P(O)(R^{k})—O—, —S—P(S)(R^{k})—O—, —S—P(O)(R^{k})—S—, —O—P(S)(R^{k})—S—. These candidates can be evaluated using methods analogous to those described above.

c. Acid Cleavable Linking Groups

In another embodiment, a cleavable linker includes an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

d. Ester-Based Linking Groups

In another embodiment, a cleavable linker includes an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

e. Peptide-Based Cleaving Groups

In yet another embodiment, a cleavable linker includes a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide-based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In one embodiment, an oligonucleotide of the invention is conjugated to a carbohydrate through a linker. Linkers include bivalent and trivalent branched linker groups. Exemplary oligonucleotide carbohydrate conjugates with linkers of the compositions and methods of the invention include, but are not limited to, those described in formulas 24-35 of PCT Publication No. WO 2018/195165.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

In certain instances, the nucleotides of an oligonucleotide can be modified by a non-ligand group. A number of non-ligand molecules have been conjugated to oligonucleotides in order to enhance the activity, cellular distribution, or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an amino linker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### IV. Pharmaceutical Compositions

The present disclosure also includes pharmaceutical compositions and formulations which include the oligonucleotides or complexes comprising two oligonucleotides of the disclosure. In one embodiment, provided herein are pharmaceutical compositions containing oligonucleotides, e.g., an oligonucleotide or complex as described herein, and a pharmaceutically acceptable carrier. The pharmaceutical compositions containing the oligonucleotides or complexes are useful for treating a subject who would benefit from editing a target gene, e.g., a polynucleotide with a SNP associated with a disease or disorder.

The pharmaceutical compositions of the present disclosure can be administered in a number of ways depending upon whether local or systemic treatment is desired and upon the area to be treated. Administration can be oral, parental, topical (e.g., by a transdermal patch), intranasal, intratracheal, epidermal and transdermal.

Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. Parenteral administration may be by continuous infusion over a selected period of time.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the oligonucleotides featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). Oligonucleotides featured in the disclosure can be encapsulated within liposomes or can form complexes thereto, in particular to cationic liposomes. Alternatively, Oligonucleotides can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-20 alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Useful solutions for oral or parenteral administration can be prepared by any of the methods well known in the pharmaceutical art, described, for example; in Remington's Pharmaceutical Sciences, 18th ed. (Mack Publishing Company, 1990). The parenteral preparation can be enclosed in ampoules, disposable syringes or multiple dose vials made of glass or plastic. Formulations also can include, for example, polyalkylene glycols such as polyethylene glycol, oils of vegetable origin, and hydrogenated naphthalenes. Other potentially useful parenteral carriers for these drugs include ethylene-vinyl acetate copolymer particles, osmotic pumps, implantable infusion systems, and liposomes.

Formulations of the present disclosure suitable for oral administration may be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug may also be administered in the form of a bolus, electuary or paste. A tablet may be made by compressing or molding the drug optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Molded tablets may be made by molding; in a suitable machine; a mixture of the powdered drug and suitable carrier moistened with an inert liquid diluent.

Oral compositions generally include an inert diluent or an edible carrier. For the purpose of oral therapeutic administration, the active compound can be incorporated with excipients. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water; ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions; methods of preparation include vacuum drying and freeze-drying which yields a powder of the active ingredient plus any additional desired ingredient from a previously sterile-filtered solution thereof.

Formulations suitable for intra-articular administration may be in the form of a sterile aqueous preparation of the drug that may be in microcrystal line form, for example, in the form of an aqueous microcrystalline suspension. Liposomal formulations or biodegradable polymer systems may also be used to present the drug for both intra-articular and ophthalmic administration.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds may be prepared with carriers that will protect the compound against rapid elimination from the body, such as a controlled release formulation, including implants and microencapsulated delivery systems. Biodegradable, biocompatible polymers can be used; such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where the active compound is to be used as part of a transplant procedure, it can be provided to the living tissue or organ to be transplanted prior to removal of tissue or organ from the donor. The compound can be provided to the donor host. Alternatively, or in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure can be formulated into any of many possible dosage forms such as, but not limited to, tablets, capsules, gel capsules, liquid syrups, soft gels, suppositories, and enemas. The compositions of the present disclosure can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizer.

The compositions of the present disclosure can also be prepared and formulated in additional formulations, such as emulsions or microemulsions, or be incorporated into a particle, e.g., a microparticle, which can be produced by spray-drying, or other methods including lyophilization, evaporation, fluid bed drying, vacuum drying, or a combination of these techniques. Penetration enhancers, e.g., surfactants, fatty acids, bile salts, chelating agents, and non-chelating non-surfactants, may be added in order to effect the efficient delivery of the compositions of the present disclosure, e.g., the delivery of the oligonucleotides, to the subject. Agents that enhance uptake of oligonucletide agents at the cellular level can also be added to the pharmaceutical and other compositions of the present disclosure, such as, cationic lipids, e.g., lipofectin, cationic glycerol derivatives, and polycationic molecules, e.g., polylysine.

The pharmaceutical composition of the present disclosure may also include a pharmaceutical carrier or excipient. A pharmaceutical carrier or excipient is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used. Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Toxicity and therapeutic efficacy of the compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). Compounds that exhibit high therapeutic indices are preferred. The data obtained from cell culture assays and animal studies can be used in formulating a range of dosage for use in humans.

The dosage of the compositions (e.g., a composition including an oligonucleotide or a complex comprising two oligonucleotides) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine whether to administer the composition and tailor the appropriate dosage and/or therapeutic regimen of treatment with the composition based on the above factors. The compositions described herein may be administered initially in a suitable dosage that may be adjusted as required, depending on the clinical response. In some embodiments, the dosage of a composition (e.g., a composition including an oligonucleotide) is a prophylactically or a therapeutically effective amount. In some embodiments, treatment of a subject with a therapeutically effective amount of a composition can include a single treatment or a series of treatments. In addition, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

The pharmaceutical compositions of the disclosure may be administered in dosages sufficient to edit a target gene, and/or treat a disease or disorder. In therapeutic use for treating, preventing, or combating, a disease or disorder, in subjects, the compounds or pharmaceutical compositions thereof will be administered orally or parenterally at a dosage to obtain and maintain a concentration, that is, an amount, or blood-level or tissue level of active component in the animal undergoing treatment which will be effective. The term “effective amount” is understood to mean that the compound of the disclosure is present in or on the recipient in an amount sufficient to elicit biological activity. Generally, an effective amount of dosage of active component will be in the range of from about 1 g/kg to about 100 mg/kg, preferably from about g/kg to about 10 mg/kg, more preferably from about 100 g/kg to about 1 mg/kg of body weight per day.

### V. Kits

In certain aspects, the instant disclosure provides kits that include a pharmaceutical formulation including an oligonucleotide agent capable of effecting an adenosine deaminase acting on RNA (ADAR)-mediated adenosine to inosine alteration of a SNP associated with a disease, and a package insert with instructions to perform any of the methods described herein.

In some embodiments, the kits include instructions for using the kit to edit a polynucleotide, e.g., a polynucleotide comprising a SNP associated with a disease or disorder. The instructions will generally include information about the use of the kit for editing nucleic acid molecules. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters.

In some embodiments, the kit includes a pharmaceutical formulation including an oligonucleotide agent or complex capable of effecting an ADAR-mediated adenosine to inosine alteration of a SNP associated with a disease, an additional therapeutic agent, and a package insert with instructions to perform any of the methods described herein.

The kit may be packaged in a number of different configurations such as one or more containers in a single box. The different components can be combined, e.g., according to instructions provided with the kit. The components can be combined according to a method described herein, e.g., to prepare and administer a pharmaceutical composition.

In some embodiments, the kit can comprise one or more containers with appropriate positive and negative controls or control samples, to be used as standard(s) for detection, calibration, or normalization.

The kit can further comprise a second container comprising a pharmaceutically-acceptable buffer, such as (sterile) phosphate-buffered saline, Ringer's solution, or dextrose solution; and other suitable additives such as penetration enhancers, carrier compounds and other pharmaceutically acceptable carriers or excipients, as described herein. It can further include other materials desirable from a commercial and user standpoint, including other buffers, diluents, filters, and package inserts with instructions for use. The kit can also include a drug delivery system such as liposomes, micelles, nanoparticles, and microspheres, as described herein. The kit can further include a delivery device, e.g., for delivery to the [central nervous system], such as needles, syringes, pumps, and package inserts with instructions for use.

This invention is further illustrated by the following examples which should not be construed as limiting. The entire contents of all references, patents and published patent applications cited throughout this application, as well as the Figures and the Sequence Listing, are hereby incorporated herein by reference.

### EXAMPLES

### Example 1: Impact of ASOs Comprising Duplex Regions on ADAR Mediated Editing Efficiency

Single stranded antisense oligonucleotides (“ASOs”) comprising duplex regions (duplex ASOs) were designed to determine the impact on recruitment and ADAR mediated editing efficiency. Such duplex ASOs can form complexes comprising two oligonucleotides wherein the duplex regions of the two oligonucleotides anneal to each other, as described herein. Editing efficiency of ASOs was tested in Alpha 1 Antitrypsin Disease ZZ Hepatocyte Like Cells (ZZ HLCs; DefiniGEN) with 1 U/μL interferon alpha at 10 and 100 nM doses.

ZZ HLCs were thawed in a 37° C. water bath in a 50 mL tube containing complete Def-Hep Thaw Medium (DTM; DefiniGEN) made according to manufacturer's instructions. After centrifugation at 100×g for 5 minutes, supernatant was aspirated, and the cell pellet was resuspended in Def-Hep Recovery and Maintenance Medium (DefiniGEN) with 1 U/mL Rock Inhibitor (Selleckchem) as per manufacturer's instructions. Cells were plated onto 384-well tissue culture treated plates at 15,000 cells per well. Cells were transferred to a hypoxic incubator (37° C., 5% CO₂, 6% O₂) and re-fed every 48-72 hours for 12-14 days with 40 μLs per well of DTM.

ASOs for transfection were prepared following a cooling protocol. Oligonucleotide solutions (100 μM in water) were heated to 95° C. using a heating block and held at 95° C. for 5 minutes. The block heating was stopped, and the oligonucleotide solutions were allowed to slowly cool down to room temperature for 1-2 hours before transfection.

On day 12, 13 or 14, ZZ HLCs were transfected with ASOs at desired concentration(s) using RNAiMax (Life Technologies, CA) according to manufacturer's protocol and placed back into the hypoxic incubator. 48 hours after transfection, mRNA was isolated from the ZZ HLCs using Oligo(dT)25 magnetic beads and relevant buffers from New England BioLabs. The samples were treated with EZ DNase (Life Technologies) after elution. The resultant isolated mRNA was used for cDNA synthesis using SuperScript IV VILO™ according to the manufacturer's instructions (Life Technologies). Ten μl of the cDNA was used for Next Generation Sequencing (NGS), Amplicon Sequencing by Quintara Biosciences.

The DNA amplicons were directly used for Amplicon Next Generation Sequencing (NGS). Percent editing of the site of Pinterest was quantified as a percentage of the number of edited nucleotides based on NGS counts. All oligonucleotides in this Example were designed for editing the same site of interest, but the different oligonucleotides incorporated a variety of modifications and a duplex region of differing length. Each oligonucleotide was assayed in at least three replicates. Assay controls lacked a duplex region and instead form a double-stranded region with a stem-loop from a single oligonucleotide.

Tables 3-5 shows percent editing of the test oligonucleotides comprising duplex regions of various length in human ZZ HLCs in the presence of interferon as measured by NGS. Data expressed as the average (Avg) of 3-4 replicates. SD=Standard deviation.

### Oligonucleotides Comprising a 26-Mer Duplex Region

TABLE 3
Editing activity for representative ASOs in human ZZ
HLCs in the presence of interferon

| | Percent Editing | | | |
|---|---|---|---|---|
| Oligo | 10 nM | | 100 nM | |
| Conc. | Mean | SD | Mean | SD |
| 26mer-1 | 5.748 | 1.669 | 14.159 | 5.784 |
| 26mer-2 | 4.332 | 2.954 | 12.862 | 6.124 |
| None | 0.117 | 0.008 | 0.159 | 0.040 |
| 26mer-2 | 0.152711 | 0.069223 | 0.251524 | 0.024381 |
| 26mer-3 | 0.126909 | 0.029136 | 0.136217 | 0.033654 |
| 26mer-4 | 0.211297 | 0.05534 | 0.660581 | 0.055168 |
| 26mer-5 | 0.317731 | 0.128776 | 1.590678 | 0.189174 |
| 26mer-6 | 0.900417 | 0.22525 | 5.058385 | 0.672022 |

### Oligonucleotides Comprising a 20-Mer Duplex Region

TABLE 4
Editing activity for representative ASOs in human
ZZ HLCs in the presence of interferon

| Edit Site | Percent Editing | | | |
|---|---|---|---|---|
| Oligo | 10 nM | | 100 nM | |
| Conc. | Mean | SD | Mean | SD |
| None | 0.117 | 0.008 | 0.159 | 0.040 |
| 20-mer 1 | 5.748 | 1.669 | 14.159 | 5.784 |
| 20-mer 2 | 4.332 | 2.954 | 12.862 | 6.124 |
| 20-mer 3 | 0.132 | 0.034 | 0.156 | 0.065 |
| 20-mer 4 | 0.850 | 0.513 | 2.501 | 0.050 |
| 20-mer 5 | 5.899 | 0.634 | 13.746 | 0.788 |
| 20-mer 6 | 4.080 | 0.679 | 7.725 | 1.105 |
| 20-mer 7 | 5.481 | 0.405 | 11.081 | 0.602 |

Oligonucleotides Comprising 20-Mer Duplex Region with Mismatches (i.e., the Double-Stranded Duplex Region was Designed to have One or More Mismatch in a Complex Comprising Two Oligonucleotides)

TABLE 5
Editing activity for representative ASOs in human ZZ
HLCs in the presence of interferon

| Edit Site | Percent Editing | | | |
|---|---|---|---|---|
| Oligo | 10 nM | | 100 nM | |
| Conc. | Mean | SD | Mean | SD |
| None | 0.117 | 0.008 | 0.159 | 0.040 |
| Mismatch | 5.748 | 1.669 | 14.159 | 5.784 |
| 20-mer 1 | | | | |
| Mismatch | 4.332 | 2.954 | 12.862 | 6.124 |
| 20-mer 2 | | | | |
| Mismatch | 0.13863775 | 0.052365 | 0.162212 | 0.04118 |
| 20-mer 3 | | | | |
| Mismatch | 0.09536625 | 0.052934 | 0.126721 | 0.026616 |
| 20-mer 4 | | | | |
| Mismatch | 0.26309325 | 0.04059 | 1.6181 | 0.352249 |
| 20-mer 5 | | | | |
| Mismatch | 2.15197 | 0.456694 | 11.93696 | 1.979011 |
| 20-mer 6 | | | | |
| Mismatch | 3.95057975 | 0.418191 | 12.76905 | 2.031541 |
| 20-mer 7 | | | | |
| Mismatch | 1.12515025 | 0.344729 | 2.783943 | 0.44147 |
| 20-mer 8 | | | | |
| Mismatch | 6.856412 | 1.090074 | 12.80491 | 1.224742 |
| 20-mer 9 | | | | |
| Mismatch | 5.43967325 | 1.634141 | 9.954537 | 0.741622 |
| 20-mer 10 | | | | |

A batch positive control was used to compare performance of the duplex ASOs. “None” is the negative control with no ASO.

As summarized in Tables 3-5, 36 to 38mer duplex oligonucleotides result in significant editing. The 18-20mer duplex region sequences within the duplex ASOs in this study showed the best editing (as shown in Tables 3 and 4 and additional data not shown), on par with or better than a non-duplex batch positive control. Additionally, activity regions that contained 2′F or LNA modifications resulted in greater editing compared to duplex ASOs with only 2′OMe modifications (data not shown).

### Example 2: Impact of Duplex ASOs Comprising Duplex Regions on ADAR-Mediated Editing Efficiency of Rab7A Site 1

Single stranded antisense oligonucleotides (“ASOs”) were designed to determine the impact of duplex ASOs on ADAR mediated editing efficiency of Rab7A. Editing efficiency of oligos was tested in Piz Mouse Hepatocytes without interferon alpha. These were tested using RNAiMAX at 10 and 100 nM, as well as without any lipofectamine at 100 and 1000 nM.

Piz Mouse Hepatocytes were thawed in a 37° C. water bath in a 50 ml tube of Cryopreserved Hepatocyte Recovery Medium (CHRM-Life Technologies). After centrifugation at 80×g for 6 minutes, supernatant was aspirated and the cell pellet is resuspended in Hepatocyte Plating Media (MB Bioscience). Cells were plated on to 384-well collagen-coated tissue culture plates at 5000 cells/well. Cells were transferred to incubator (37° C.), 4 to 6 hours later media was changed to Hepatocyte Maintenance Media (MB Bioscience) and cells were transfected with ASOs at desired concentrations, with and without RNAiMax (Life Technologies, CA) according to manufacturer's protocol and placed back into the incubator. Further processing was conducted as described in Example 1.

The below DNA amplicons were directly used for Amplicon Next Generation Sequencing (NGS). Percent editing was quantified as a percentage of the number of G vs A nucleotides at the Rab7 site based on NGS counts. Each oligonucleotide was assayed in at least three replicates. Primer sequences used for NGS are shown in Table 6.

TABLE 6
PCR and sequencing primers

| | | |
|---|---|---|
| SEQ ID | Forward primer Mouse Rab7 | gaattcctggatt |
| NO: 1 | Site 1 | gtgtgtttaagt |
| SEQ ID | Reverse primer Mouse Rab7 | cagaagggaactg |
| NO: 2 | Site 1 | tctagttcagt |

The following abbreviations are used to indicate modifications in the oligonucleotide sequences.

| Modification | Abbreviation |
|---|---|
| RNA | rN |
| DNA | dN |
| 2′-O-Methyl(2′-OMe) | mN |
| 2′-F-RNA | FN |
| Phosphorothioate | * |
| internucleoside linkage | |
| LNA | L |
| PO-Galnac | Tri-GalNAc |
| {mismatch} | Mismatch |

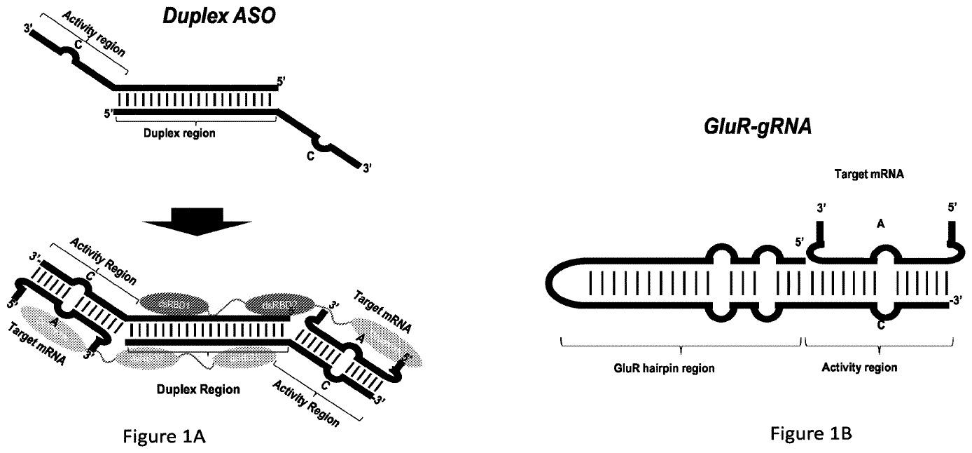

In the duplex ASO sequences shown in all the Examples herein, the duplex region sequence is delineated from the activity region sequence (see FIG. 1) by an underscore (_). The duplex region starts from the 5′ end of the ASO and ends 6 nucleotides 5′ of the DNA triplet (triplet shown in bold for example, as dCdCdA in Table 8 linked by phosphorothioate internucleoside linkages). However, the present embodiments are not limited to this representative placement of the duplex region within the duplex ASO. The 2′F positions are also bolded for easy identification. The nucleotides from after the dash to the 3′ end of the ASO form the activity region.

TABLE 7
Assay Control ASOs

| | | |
|---|---|---|
| SEQ ID | KB007102 | 5′- |
| NO: 3 | -2 | mC*mA*mU*mA*FA*mUmU*mCmU*mUFG*mUFG*mUFC*mUFA*FC*mUFG* |
| | | mUFA*FC*FA*FG*FC*FA*mUFA*FC*FU*mG*mCFC*mGdC*dC*dA*mGFC* |
| | | mUmG*mGmA*mUmU*mU*mC*mC*mC-3′-PO-GalNAc |
| SEQ ID | KB007254 | 5′- |
| NO: 4 | -2 | mG*mU*mG*mU*FC*mU*mAFC*mUmG*mUFA*FC*mA*mGmC*mAFU*mAF |
| | | C*FU*mG*mCFC*mGdC*dC*dA*mGFC*mUmG*FG*FA*mUFU*mUmG*mCm |
| | | C*FA*FA*mUmU*mCFU*mGmA*FG*mU*mA*mA*mC-3′-PO-GalNAc |

TABLE 8
Duplex ASOs Targeting Human Rab7A Site 1

| SEQ ID | | |
|---|---|---|
| NO | Oligo# | |
| | | Rab7a Site 1: 26-mer duplex region |
| 5 | KB012536-1 | 5′- |
| | | rG*rG*rUrGrUrCrGrArGrGrArGrUrArCrUrCrCrUrCrGrArCrArCrC_mCmUmG |
| | | mCmCmGdC*dC*dA*mGmCmUmGmG*mA*mU*mU*mU-3′-PO-GalNAc |
| 6 | KB012537-1 | 5′- |
| | | FG*FG*mUFG*mUmCFG*FA*FG*FG*FA*FG*mUFA*mCmUmCmCmUmCF |
| | | G*FA*mCFA*mCmC_mCmUmGmCmCmGdC*dC*dA*mGmCmUmGmG*mA |
| | | *mU*mU*mU-3′-PO-GalNAc |
| 7 | KB012538-1 | 5′- |
| | | FG*FG*mUFG*mUmCFG*FA*FG*FG*FA*FG*mUFA*mCmUmCmCmUmCF |
| | | G*FA*mCFA*mCmC_FC*FU*mGmCFC*mGdC*dC*dA*mGFC*mUmGFG*F |
| | | A*mU*mU*mU-3′-PO-GalNAc |
| 8 | KB012539-1 | 5′- |
| | | FG*FG*mUFG*mUmCFG*FA*FG*FG*FA*FG*mUFA*mCmUmCmCmUmCF |
| | | G*FA*mCFA*mCmC_mC*LTmG*mCmC*mGdC*dC*dA*mGmC*mUmG*LG* |
| | | mA*mU*LT*mU-3′-PO-GalNAc |
| 9 | KB012540-1 | 5′- |
| | | FG*FG*mUFG*mUmCFG*FA*FG*FG*FA*FG*mUFA*mCmUmCmCmUmCF |
| | | G*FA*mCFA*mCmC_FC*LTmG*mCFC*mGdC*dC*dA*mGFC*mUmG*LG*F |
| | | A*mU*LT*mU-3′-PO-GalNAc |
| | | Rab7a Site 1: 20-mer duplex region |
| 10 | KB012541-1 | 5′- |
| | | rG*rG*rUrGrUrCrGrArGrGrCrCrUrCrGrArCrArCrC_mCmUmGmCmCmGdC* |
| | | dC*dA*mGmCmUmGmG*mA*mU*mU*mU-3′-PO-GalNAc |
| 11 | KB012542-1 | 5′- |
| | | FG*FG*mUFG*mUmCFG*FA*FG*FG*mCmCmUmCFG*FA*mCFA*mCmC_ |
| | | mCmUmGmCmCmGdC*dC*dA*mGmCmUmGmG*mA*mU*mU*mU-3′-PO- |
| | | GalNAc |
| 12 | KB012543-1 | 5′- |
| | | FG*FG*mUFG*mUmCFG*FA*FG*FG*mCmCmUmCFG*FA*mCFA*mCmC_F |
| | | C*FU*mGmCFC*mGdC*dC*dA*mGFC*mUmGFG*FA*mU*mU*mU-3′-PO- |
| | | GalNAc |
| 13 | KB012544-1 | 5′- |
| | | FG*FG*mUFG*mUmCFG*FA*FG*FG*mCmCmUmCFG*FA*mCFA*mCmC_ |
| | | mC*LTmG*mCmC*mGdC*dC*dA*mGmC*mUmG*LG*mA*mU*LT*mU-3′- |
| | | PO-GalNAc |
| 14 | KB012545-1 | 5′- |
| | | FG*FG*mUFG*mUmCFG*FA*FG*FG*mCmCmUmCFG*FA*mCFA*mCmC_F |
| | | C*LTmG*mCFC*mGdC*dC*dA*mGFC*mUmG*LG*FA*mU*LT*mU-3′-PO- |
| | | GalNAc |
| | | Rab7a Site 1: 20-mer duplex region with mismatches |
| 15 | KB012546-1 | 5′- |
| | | rG*rG*rUrG{mismatch}rCrCrGrArGrGrCrCrUrCrGrArCrArCrC_mCm |
| | | UmGmCmCmGdC*dC*dA*mGmCmUmGmG*mA*mU*mU*mU-3′-PO-GalNAc |
| 16 | KB012547-1 | 5′- |
| | | rG*rG*rUrGrCrGrArGrGrCrCrU{mismatch}rGrGrArCrArCrC_mCmU |
| | | mGmCmCmGdC*dC*dA*mGmCmUmGmG*mA*mU*mU*mU-3′-PO-GalNAc |
| 17 | KB012548-1 | 5′- |
| | | FG*FG*mUFG*{mismatch}mCmCFG*FA*FG*FG*mCmCmUmCFG*FA*mC |
| | | FA*mCmC_mCmUmGmCmCmGdC*dC*dA*mGmCmUmGmG*mA*mU*mU* |
| | | mU-3′-PO-GalNAc |
| 18 | KB012549-1 | 5′- |
| | | FG*FG*mUFG*{mismatch}mCmCFG*FA*FG*FG*mCmCmUmCFG*FA*mC |
| | | FA*mCmC_FC*FU*mGmCFC*mGdC*dC*dA*mGFC*mUmGFG*FA*mU*mU* |
| | | mU-3′-PO-GalNAc |
| 19 | KB012550-1 | 5′- |
| | | FG*FG*mUFG*{mismatch}mCmCFG*FA*FG*FG*mCmCmUmCFG*FA*mC |
| | | FA*mCmC_FC*LTmG*mCFC*mGdC*dC*dA*mGFC*mUmG*LG*FA*mU*LT* |
| | | mU-3′-PO-GalNAc |
| 20 | KB012551-1 | 5′- |
| | | FG*FG*mUFG*mUmCFG*FA*FG*FG*mCmCmU{mismatch}mGFG*FA*mC |
| | | FA*mCmC_mCmUmGmCmCmGdC*dC*dA*mGmCmUmGmG*mA*mU*mU* |
| | | mU-3′-PO-GalNAc |
| 21 | KB012552-1 | 5′- |
| | | FG*FG*mUFG*mUmCFG*FA*FG*FG*mCmCmU{mismatch}mGFG*FA*mC |
| | | FA*mCmC_FC*FU*mGmCFC*mGdC*dC*dA*mGFC*mUmGFG*FA*mU*mU* |
| | | mU-3′-PO-GalNAc |
| 22 | KB012553-1 | 5′- |
| | | FG*FG*mUFG*mUmCFG*FA*FG*FG*mCmCmU{mismatch}mGFG*FA*mC |
| | | FA*mCmC_FC*LTmG*mCFC*mGdC*dC*dA*mGFC*mUmG*LG*FA*mU*LT* |
| | | mU-3′-PO-GalNAc |

The results are shown in Tables 9-11.

### Rab7a Site 1: 26-Mer Duplex Region

TABLE 9
Editing activity for ASOs shown in Table 8 in Piz Mouse Hepatocytes with and
without RNAiMax

| | Free Uptake | | | | RNAiMax | | | |
|---|---|---|---|---|---|---|---|---|
| Oligo Conc. | 100 nM | | 1000 nM | | 10 nM | | 100 nM | |
| None | 0.331 | 0.193 | 0.240 | 0.037 | 0.225 | 0.038 | 0.246 | 0.059 |
| KB007102-2 | 17.368 | 3.724 | 14.149 | 2.053 | 75.407 | 4.158 | 68.398 | 2.494 |
| KB007254-2 | 6.326 | 0.914 | 4.837 | 0.935 | 74.469 | 2.863 | 74.378 | 1.094 |
| KB012536-1 | 0.913 | 0.820 | 0.612 | 0.612 | 6.373 | 0.728 | 13.751 | 4.178 |
| KB012537-1 | 1.535 | 0.648 | 2.504 | 1.634 | 46.535 | 3.458 | 58.769 | 8.980 |
| KB012538-1 | 1.689 | 0.710 | 2.647 | 1.224 | 52.034 | 6.208 | 46.897 | 24.499 |
| KB012539-1 | 1.005 | 0.152 | 1.161 | 0.158 | 31.103 | 2.808 | 37.816 | 4.258 |
| KB012540-1 | 1.447 | 0.285 | 1.657 | 0.299 | 43.663 | 2.417 | 50.858 | 5.537 |

### Rab7a Site 1: 20-Mer Duplex Region

TABLE 10
Editing activity for ASOs shown in Table 8 in Piz Mouse Hepatocytes with and
without RNAiMax

| | Free Uptake | | | | RNAiMax | | | |
|---|---|---|---|---|---|---|---|---|
| Oligo Conc. | 100 nM | | 1000 nM | | 10 nM | | 100 nM | |
| None | 0.331 | 0.193 | 0.240 | 0.037 | 0.225 | 0.038 | 0.246 | 0.059 |
| KB007102-2 | 17.368 | 3.724 | 14.149 | 2.053 | 75.407 | 4.158 | 68.398 | 2.494 |
| KB007254-2 | 6.326 | 0.914 | 4.837 | 0.935 | 74.469 | 2.863 | 74.378 | 1.094 |
| KB012541-1 | 0.229 | 0.048 | 0.323 | 0.057 | 12.885 | 2.570 | 38.773 | 7.729 |
| KB012542-1 | 7.266 | 0.987 | 6.615 | 0.896 | 65.299 | 6.904 | 64.965 | 6.053 |
| KB012543-1 | 4.423 | 1.421 | 5.164 | 0.786 | 65.763 | 7.825 | 72.083 | 10.073 |
| KB012544-1 | 2.866 | 0.966 | 3.113 | 2.090 | 39.178 | 20.057 | 31.560 | 20.004 |
| KB012545-1 | 3.455 | 0.918 | 3.921 | 0.709 | 48.870 | 2.064 | 45.513 | 4.960 |

Rab7a Site 1: 20-Mer Duplex Region with Mismatches

TABLE 11
Editing activity for ASOs shown in Table 8 in Piz Mouse Hepatocytes with and
without RNAiMax.

| | Free Uptake | | | | RNAiMax | | | |
|---|---|---|---|---|---|---|---|---|
| Oligo Conc. | 100 nM | | 1000 nM | | 10 nM | | 100 nM | |
| None | 0.331 | 0.193 | 0.240 | 0.037 | 0.225 | 0.038 | 0.246 | 0.059 |
| KB007102-2 | 17.368 | 3.724 | 14.149 | 2.053 | 75.407 | 4.158 | 68.398 | 2.494 |
| KB007254-2 | 6.326 | 0.914 | 4.837 | 0.935 | 74.469 | 2.863 | 74.378 | 1.094 |
| KB012546-1 | 1.24 | 1.50 | 1.62 | 1.54 | 6.91 | 1.35 | 26.04 | 7.67 |
| KB012547-1 | 0.26 | 0.08 | 0.18 | 0.12 | 0.97 | 0.19 | 5.68 | 1.71 |
| KB012548-1 | 4.08 | 0.25 | 5.18 | 1.62 | 52.31 | 3.77 | 68.04 | 3.97 |
| KB012549-1 | 3.41 | 0.95 | 3.08 | 0.51 | 60.70 | 1.73 | 75.71 | 4.86 |
| KB012550-1 | 1.76 | 0.51 | 1.60 | 0.66 | 51.23 | 3.37 | 41.55 | 9.83 |
| KB012551-1 | 9.85 | 1.63 | 8.24 | 1.38 | 64.42 | 10.67 | 65.28 | 6.49 |
| KB012552-1 | 5.13 | 2.87 | 2.93 | 3.15 | 43.62 | 29.81 | 43.11 | 31.45 |
| KB012553-1 | 4.53 | 0.69 | 3.87 | 0.88 | 49.62 | 2.31 | 45.10 | 4.05 |

ASOs KB007102-2 and KB007254-2 were used as positive assay controls, and “None” is the negative control. As shown in Tables 9-11, the 38mer duplex oligonucleotides perform well in both transfection and free uptake. It was observed that 20mer duplex sequences performed better overall when compared to 26mer duplex sequences. Furthermore, in contrast to Example 1, a 2′-OMe modified activity region edited better than certain other modified sequences tested.

### Example 3: Impact of ASOs Comprising Duplex Regions on ADAR-Mediated Editing Efficiency of Cyno-SERPINA1-K359E

Single stranded antisense oligonucleotides (“ASOs”) were designed to interrogate the impact of duplex ASOs on ADAR mediated editing efficiency of a third target, Cyno SerpinA1-K359E. Editing efficiency for oligos was tested in Piz Mouse Hepatocytes without interferon alpha. These were tested using RNAiMAX at 10 and 100 nM, as well as without any lipofectamine at 100 and 1000 nM.

Transfection and cell protocols were substantially the same as Example 2 above.

The below DNA amplicons were directly used for Amplicon Next Generation Sequencing (NGS). Percent editing was quantified as a percentage of the number of G vs A nucleotides at the K359E site based on NGS counts. Each oligonucleotide was assayed in at least three replicates. Primer sequences used for NGS are shown in Table 12.

TABLE 12
PCR and sequencing primers

| | | |
|---|---|---|
| SEQ ID | Forward primer Cyno | ACCTGGGTATCACT |
| NO: 23 | SERPINA1- K359E | AAGGTCTTCA |
| SEQ ID | Reverse primer Cyno | GTTCAATCATTAAG |
| NO: 24 | SERPINA1- K359E | AAGACAAAGGGT |

The following abbreviations are used to indicate modifications in the oligonucleotide sequences.

| Modification | Abbreviation |
|---|---|
| RNA | rN |
| DNA | dN |
| 2′-O-Methyl(2′-OMe) | mN |
| 2′-F-RNA | FN |
| Phosphorothioate | * |
| internucleoside linkage | |
| LNA | LN |
| PO-Galnac | Tri-GalNAc |
| {mismatch} | Mismatch |

TABLE 13
Assay Controls

| | | |
|---|---|---|
| SEQ | KB00 | 5′- |
| ID | 6522 | mG*FG*mC*FC*FC*mCmAFG*mCFA*mGFC*FU*mUmCmAFG*FU*mCFC*FC*mU |
| NO: | -2 | mUFU*mCdT*dC*dG*mUFC*mGmAFU*FG*mGFU*FC*mAmGmCFA*FC*mAFG*m |
| 25 | | CFC*mUmU*FA*FU*mG*FC*mA-3′ |
| SEQ | KB00 | 5′- |
| ID | 7102 | mC*mA*mU*mA*FA*mUmU*mCmU*mUFG*mUFG*mUFC*mUFA*FC*mUFG*mUFA |
| NO: | -1 | *FC*FA*FG*FC*FA*mUFA*FC*FU*mG*mCFC*mGdC*dC*dA*mGFC*mUmG*mGm |
| 26 | | A*mUmU*mU*mC*mC*mC-3′-PO-GalNAc |

TABLE 14
Duplex ASOs Targeting Cyno SERPINA1- K359E

| SEQ | Oligo | |
|---|---|---|
| ID NO | # | |
| | | Cyno-SERPINA1-K359E: 26-mer duplex region |
| 27 | KB01 | 5′- |
| | 2579- | rG*rG*rUrGrUrCrGrArGrGrArGrUrArCrUrCrCrUrCrGrArCrArCrC_mAmCmAmG |
| | 1 | mCmCdT*dC*dA*mUmGmCmAmC*mG*mG*mC*mC-3′-PO-GalNAc |
| 28 | KB01 | 5′- |
| | 2580- | FG*FG*mUFG*mUmCFG*FA*FG*FG*FA*FG*mUFA*mCmUmCmCmUmCFG*FA*mCF |
| | 1 | A*mCmC_mAmCmAmGmCmCdT*dC*dA*mUmGmCmAmC*mG*mG*mC*mC-3′-PO- |
| | | GalNAc |
| 29 | KB01 | 5′- |
| | 2581- | FG*FG*mUFG*mUmCFG*FA*FG*FG*FA*FG*mUFA*mCmUmCmCmUmCFG*FA*mCF |
| | 1 | A*mCmC_FA*FC*mAmGFC*mCdT*dC*dA*mUFG*mCmAFC*FG*mG*mC*mC-3′-PO- |
| | | GalNAc |
| 30 | KB01 | 5′- |
| | 2582- | FG*FG*mUFG*mUmCFG*FA*FG*FG*FA*FG*mUFA*mCmUmCmCmUmCFG*FA*mCF |
| | 1 | A*mCmC_mA*LCmA*mGmC*mCdT*dC*dA*mUmG*mCmA*LC*mG*mG*LC*mC-3′- |
| | | PO-GalNAc |
| 31 | KB01 | 5′- |
| | 2583- | FG*FG*mUFG*mUmCFG*FA*FG*FG*FA*FG*mUFA*mCmUmCmCmUmCFG*FA*mCF |
| | 1 | A*mCmC_FA*LCmA*mGFC*mCdT*dC*dA*mUFG*mCmA*LC*FG*mG*LC*mC-3′-PO- |
| | | GalNAc |
| | | Cyno-SERPINA1-K359E: 20-mer duplex region |
| 32 | KB01 | 5′- |
| | 2584- | rG*rG*rUrGrUrCrGrArGrGrCrCrUrCrGrArCrArCrC_mAmCmAmGmCmCdT*dC* |
| | 1 | dA*mUmGmCmAmC*mG*mG*mC*mC-3′-PO-GalNAc |
| 33 | KB01 | 5′- |
| | 2585- | FG*FG*mUFG*mUmCFG*FA*FG*FG*mCmCmUmCFG*FA*mCFA*mCmC_mAmCmA |
| | 1 | mGmCmCdT*dC*dA*mUmGmCmAmC*mG*mG*mC*mC-3′-PO-GalNAc |
| 34 | KB01 | 5′- |
| | 2586- | FG*FG*mUFG*mUmCFG*FA*FG*FG*mCmCmUmCFG*FA*mCFA*mCmC_FA*FC*mA |
| | 1 | mGFC*mCdT*dC*dA*mUFG*mCmAFC*FG*mG*mC*mC-3′-PO-GalNAc |
| 35 | KB01 | 5′- |
| | 2587- | FG*FG*mUFG*mUmCFG*FA*FG*FG*mCmCmUmCFG*FA*mCFA*mCmC_mALCmAm |
| | 1 | GmCmCdT*dC*dA*mUmGmCmALC*mG*mG*LC*mC-3′-PO-GalNAc |
| 36 | KB01 | 5′- |
| | 2588- | FG*FG*mUFG*mUmCFG*FA*FG*FG*mCmCmUmCFG*FA*mCFA*mCmC_FA*LCmA |
| | 1 | mGFC*mCdT*dC*dA*mUFG*mCmALC*FG*mG*LC*mC-3′-PO-GalNAc |
| | | Cyno-SERPINA1-K359E: 20-mer duplex regions with mismatches |
| 37 | KB01 | 5′- |
| | 2589- | rG*rG*rUrG{mismatch}rCrCrGrArGrGrCrCrUrCrGrArCrArCrCmAmCmAmGm |
| | 1 | CmCdT*dC*dA*mUmGmCmAmC*mG*mG*mC*mC-3′-PO-GalNAc |
| 38 | KB01 | 5′- |
| | 2590- | rG*rG*rUrGrUrCrGrArGrGrCrCrU{mismatch}rGrGrArCrArCrCmAmCmAmGm |
| | 1 | CmCdT*dC*dA*mUmGmCmAmC*mG*mG*mC*mC-3′-PO-GalNAc |
| 39 | KB01 | 5′- |
| | 2591- | FG*FG*mUFG*{mismatch}mCmCFG*FA*FG*FG*mCmCmUmCFG*FA*mCFA*mCmC |
| | 1 | _mAmCmAmGmCmCdT*dC*dA*mUmGmCmAmC*mG*mG*mC*mC-3′-PO-GalNAc |
| 40 | KB01 | 5′- |
| | 2592- | FG*FG*mUFG*{mismatch}mCmCFG*FA*FG*FG*mCmCmUmCFG*FA*mCFA*mCmC |
| | 1 | _FA*FC*mAmGFC*mCdT*dC*dA*mUFG*mCmAFC*FG*mG*mC*mC-3′-PO-GalNAc |
| 41 | KB01 | 5′- |
| | 2593- | FG*FG*mUFG*{mismatch}mCmCFG*FA*FG*FG*mCmCmUmCFG*FA*mCFA*mCmC |
| | 1 | _FA*LCmAmGFC*mCdT*dC*dA*mUFG*mCmALC*FG*mG*LC*mC-3′-PO-GalNAc |
| 42 | KB01 | 5′- |
| | 2594- | FG*FG*mUFG*mUmCFG*FA*FG*FG*mCmCmU{mismatch}mGFG*FA*mCFA*mCmC |
| | 1 | _mAmCmAmGmCmCdT*dC*dA*mUmGmCmAmC*mG*mG*mC*mC-3′-PO-GalNAc |
| 43 | KB01 | 5′- |
| | 2595- | FG*FG*mUFG*mUmCFG*FA*FG*FG*mCmCmU{mismatch}mGFG*FA*mCFA*mCmC |
| | 1 | _FA*FC*mAmGFC*mCdT*dC*dA*mUFG*mCmAFC*FG*mG*mC*mC-3′-PO-GalNAc |
| 44 | KB01 | 5′- |
| | 2596- | FG*FG*mUFG*mUmCFG*FA*FG*FG*mCmCmU{mismatch}mGFG*FA*mCFA*mCmC |
| | 1 | _FA*LCmAmGFC*mCdT*dC*dA*mUFG*mCmALC*FG*mG*LC*mC-3′-PO-GalNAc |

The results are shown in Tables 15-17.

Cyno-SERPINA1-K359E: 26-mer Duplex Region

TABLE 15
Editing activity for ASOs shown in Table 14 in Piz Mouse Hepatocytes with and
without RNAiMax

| | Free Uptake | | | | RNAiMax | | | |
|---|---|---|---|---|---|---|---|---|
| Oligo Conc. | 100 nM | | 1000 nM | | 10 nM | | 100 nM | |
| None | 0.128 | 0.020 | 0.179 | 0.055 | 0.136 | 0.022 | 0.132 | 0.033 |
| KB007102-2 | 17.368 | 3.724 | 14.149 | 2.053 | 75.407 | 4.158 | 68.398 | 2.494 |
| KB006522-2 | 2.575 | 1.449 | 4.431 | 0.370 | 47.013 | 4.120 | 75.463 | 3.446 |
| KB012579-1 | 0.156 | 0.038 | 0.227 | 0.091 | 3.918 | 0.895 | 10.014 | 1.986 |
| KB012580-1 | 0.3779 | 0.0410 | 0.3607 | 0.0570 | 8.1097 | 1.0373 | 9.1219 | 1.1827 |
| KB012581-1 | 1.2614 | 0.2875 | 1.9574 | 0.3802 | 32.3274 | 2.1304 | 41.3717 | 2.2741 |
| KB012582-1 | 0.4504 | 0.1068 | 0.2547 | 0.0556 | 3.9850 | 0.7823 | 3.7915 | 0.3801 |
| KB012583-1 | 0.6151 | 0.1948 | 0.5983 | 0.0560 | 18.0742 | 1.9593 | 19.0672 | 1.3799 |

Cyno-SERPINA1-K359E: 20-mer duplex region

TABLE 16
Editing activity for ASOs shown in Table 14 in Piz Mouse Hepatocytes with and
without RNAiMax

| | Free Uptake | | | | RNAiMax | | | |
|---|---|---|---|---|---|---|---|---|
| Oligo Conc. | 100 nM | | 1000 nM | | 10 nM | | 100 nM | |
| None | 0.128 | 0.020 | 0.179 | 0.055 | 0.136 | 0.022 | 0.132 | 0.033 |
| KB007102-2 | 17.368 | 3.724 | 14.149 | 2.053 | 75.407 | 4.158 | 68.398 | 2.494 |
| KB006522-2 | 2.575 | 1.449 | 4.431 | 0.370 | 47.013 | 4.120 | 75.463 | 3.446 |
| KB012584-1 | 0.107 | 0.022 | 0.124 | 0.051 | 6.536 | 1.001 | 14.426 | 2.691 |
| KB012585-1 | 1.912 | 0.259 | 1.467 | 0.373 | 29.943 | 1.668 | 32.781 | 2.785 |
| KB012586-1 | 3.857 | 0.219 | 3.731 | 0.583 | 47.867 | 7.897 | 62.816 | 5.233 |
| KB012587-1 | 1.190 | 0.110 | 1.213 | 0.142 | 18.413 | 4.320 | 14.266 | 0.831 |
| KB012588-1 | 2.572 | 0.452 | 2.045 | 0.397 | 33.410 | 2.292 | 35.309 | 2.896 |

Cyno-SERPINA1-K359E: 20-Mer Duplex Regions with Mismatches

TABLE 17
Editing activity for ASOs shown in Table 14 in Piz Mouse Hepatocytes with and
without RNAiMax

| | Free Uptake | | | | RNAiMax | | | |
|---|---|---|---|---|---|---|---|---|
| Oligo Conc. | 100 nM | | 1000 nM | | 10 nM | | 100 nM | |
| None | 0.128 | 0.020 | 0.179 | 0.055 | 0.136 | 0.022 | 0.132 | 0.033 |
| KB007102-2 | 17.368 | 3.724 | 14.149 | 2.053 | 75.407 | 4.158 | 68.398 | 2.494 |
| KB006522-2 | 2.575 | 1.449 | 4.431 | 0.370 | 47.013 | 4.120 | 75.463 | 3.446 |
| KB012589-1 | 0.142 | 0.061 | 0.118 | 0.040 | 3.790 | 0.253 | 14.859 | 2.575 |
| KB012590-1 | 0.132 | 0.031 | 0.137 | 0.030 | 0.787 | 0.222 | 2.953 | 0.932 |
| KB012591-1 | 0.539 | 0.113 | 0.462 | 0.050 | 18.515 | 0.371 | 32.006 | 0.539 |
| KB012592-1 | 1.836 | 0.549 | 1.504 | 0.129 | 37.377 | 3.157 | 65.727 | 1.571 |
| KB012593-1 | 0.730 | 0.139 | 1.001 | 0.278 | 35.934 | 3.491 | 40.848 | 4.678 |
| KB012594-1 | 1.274 | 0.147 | 1.094 | 0.183 | 21.206 | 4.455 | 29.383 | 4.828 |
| KB012595-1 | 3.732 | 0.553 | 3.504 | 0.502 | 32.320 | 1.651 | 47.517 | 3.404 |
| KB012596-1 | 2.116 | 0.267 | 1.838 | 0.437 | 28.874 | 4.050 | 30.233 | 1.187 |

ASO KB006522 was used as a batch control to compare performance of the duplex ASOs. ASO KB007102 was used as positive assay control, and “None” is the negative control. As shown in Tables 15-17, a 38mer duplex ASO containing a 20mer duplex region showed >65% editing when transfected, which was better than a control editing oligonucleotide (53mer) that lacks a duplex region. 20mer duplex regions performed better than 26mer duplex regions overall, which is similar to what was seen in Example 2 (Rab7A Site 1). From the experiments on duplex region length, the highest editing was seen with ASOs having an 18-20mer duplex sequence. Editing decreased with decreasing length of duplex until no editing was seen with a 12mer duplex region in these experiments. An activity region with 2′F modifications showed better editing than LNA in both transfection and free uptake.

These studies demonstrated that duplex region structure is important for ADAR recruitment and editing. Editing was achieved in two cell types at different concentrations. Thus, a single-stranded ASO comprising a duplex region (i.e., duplex ASO) can be an easier, more efficient therapeutic to produce and deliver ASOs in vivo compared to longer oligonucleotides such as GluR-based guide comprising a stem-loop structure, as duplex ASOs can mediate ADAR recruitment and in vivo editing.

### EQUIVALENTS

Those skilled in the art will recognize, or be able to ascertain, using no more than routine experimentation, many equivalents to the specific embodiments and methods described herein. Such equivalents are intended to be encompassed by the scope of the following claims.

We claim:

1. An oligonucleotide comprising an activity region and a duplex region, wherein
a) the activity region comprises the structure: 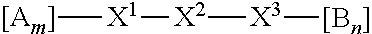
wherein each of A and B is a nucleotide;
m and n are each, independently, an integer from 2 to 50;
X¹, X², and X³ are each, independently, a nucleotide; and
b) the duplex region consists of 8-30 nucleotides, wherein the nucleobase sequence of the duplex region is substantially reverse complementary to itself.

2. The oligonucleotide of claim 1, wherein the duplex region comprises 15-30, 14-29, 13-28, 12-27, 11-26, 10-25, 9-24, 8-23, 12-28, 13-27, 14-26, 15-25, 16-24, 17-23, 18-22, or 19-21 nucleotides.

3. The oligonucleotide of claim 1 or claim 2, wherein the activity region comprises 15-30, 14-29, 13-28, 12-27, 11-26, 10-25, 9-24, 8-23, 12-28, 13-27, 14-26, 15-25, 16-24, 17-23, 18-22, or 19-21 nucleotides.

4. The oligonucleotide of any one of claims 1-3, wherein the duplex region is 5′ of the activity region.

5. The oligonucleotide of any one of claims 1-3, wherein the duplex region is 3′ of the activity region.

6. The oligonucleotide of any one of claims 1-5, wherein the nucleotides of the duplex region are each independently selected from a 2′-O—C₁-C₆ alkyl-nucleotide, a 2′-amino-nucleotide, an arabino nucleic acid-nucleotide, a bicyclic-nucleotide, a 2′-O-methoxyethyl-nucleotide, a constrained ethyl (cEt)-nucleotide, a LNA-nucleotide, a ribonucleotide, and a DNA-nucleotide.

7. The oligonucleotide of any one of claims 1-6, wherein at physiological conditions, the duplex regions of two oligonucleotides form a double-stranded region.

8. The oligonucleotide of claim 7, wherein the double-stranded region formed by the duplex region of two oligonucleotides is 8-30 base pairs long, including any mismatches.

9. The oligonucleotide of claim 7 or claim 8, wherein the double-stranded region formed by the duplex region of two oligonucleotides comprises 0, 1, 2, 3, 4, or 5 mismatches.

10. The oligonucleotide of any one of claims 7-9, wherein the double-stranded region formed by the duplex regions of two oligonucleotides comprises 11-30 base pairs or 18-26 base pairs, including any mismatches.

11. The oligonucleotide of any one of claims 7-10, wherein the double-stranded regions formed by the duplex regions of two oligonucleotides is capable of recruiting an adenosine deaminase acting on RNA (ADAR) enzyme.

12. The oligonucleotide of any one of claims 1-11, wherein the oligonucleotide consists of 20-80 nucleotides, or 20-70 nucleotides, or 20-60 nucleotides, or 30-60 nucleotides, or 30-50 nucleotides.

13. The oligonucleotide of any one of claims 1-12, wherein the activity region is substantially complementary to a target mRNA.

14. The oligonucleotide of any one of claims 1-13, wherein at least one nucleotide of A and/or B is a 2′-F-nucleotide, optionally wherein at least one 2′-F-nucleotide is at a position selected from +8, +3, −3, −7, −19 and −22, wherein X² is position 0, X¹ is position −1, and X³ is position +1.

15. The oligonucleotide of any one of claims 1-14, wherein all purines comprised in the duplex region are 2′-fluoro nucleotides and all pyrimidines comprised in the duplex region are 2′-O-methoxyethyl-nucleotides.

16. The oligonucleotide of any one of claims 14-15, wherein the remaining nucleotides of [Aₘ] are each independently selected from a 2′-O—C₁-C₆ alkyl-nucleotide, a 2′-amino-nucleotide, an arabino nucleic acid-nucleotide, a bicyclic-nucleotide, a 2′-O-methoxyethyl-nucleotide, a constrained ethyl (cEt)-nucleotide, a LNA-nucleotide, a ribonucleotide, and a DNA-nucleotide.

17. The oligonucleotide of any one of claims 14-15, wherein the remaining nucleotides of [Aₘ] are each independently selected from a 2′-O-methyl-nucleotide, a 2′-F-nucleotide, a 2′-O methoxyethyl-nucleotide, a cEt-nucleotide, a LNA-nucleotide, a ribonucleotide, and a DNA-nucleotide.

18. The oligonucleotide of any one of claims 14-15, wherein the remaining nucleotides of [Aₘ] are 2′-O-methyl-nucleotides.

19. The oligonucleotide of any one of claims 1-18, wherein [Aₘ] comprises at least one phosphorothioate linkage.

20. The oligonucleotide of any one of claims 1-19, wherein [Aₘ] comprises at least four terminal phosphorothioate linkages.

21. The oligonucleotide of any one of claims 19-20, wherein at least one phosphorothioate linkage is stereopure.

22. The oligonucleotide of any one of claims 1-21, wherein [Bₙ] comprises at least one nuclease resistant nucleotide.

23. The oligonucleotide of any one of claims 1-22, wherein each nucleotide of [Bₙ] is a nuclease resistant nucleotide.

24. The oligonucleotide of any one of claims 1-23, wherein each nucleotide of [Bₙ] is independently selected from a 2′-O—C₁-C₆ alkyl-nucleotide, a 2′-amino-nucleotide, an arabino nucleic acid-nucleotide, a bicyclic-nucleotide, a 2′-O-methoxyethyl-nucleotide, a constrained ethyl (cEt)-nucleotide, a LNA-nucleotide, a ribonucleotide, and a DNA-nucleotide.

25. The oligonucleotide of any one of claims 1-24, wherein each nucleotide of [Bₙ] is independently selected from a 2′-O-methyl-nucleotide, a 2′-O-methoxyethyl-nucleotide, a cEt-nucleotide, a LNA-nucleotide, a ribonucleotide, and a DNA-nucleotide.

26. The oligonucleotide of any one of claims 1-25, wherein each nucleotide of [Bₙ] is a 2′-O-methyl-nucleotide.

27. The oligonucleotide of any one of claims 1-26, wherein [Bₙ] comprises at least one phosphorothioate linkage.

28. The oligonucleotide of any one of claims 1-27, wherein [Bₙ] comprises at least four terminal phosphorothioate linkages.

29. The oligonucleotide of any one of claims 27-28, wherein at least one phosphorothioate linkage is stereopure.

30. The oligonucleotide of any one of claims 1-29, wherein the oligonucleotide comprises 1, 2, 3, or 4 2′-F-nucleotides.

31. The oligonucleotide of any one of claims 1-30, wherein X² is not a 2′-O-methyl-nucleotide.

32. The oligonucleotide of any one of claims 1-31, wherein X¹, X², and X³ are not 2′-O-methyl-nucleotides.

33. The oligonucleotide of any one of claims 1-32, wherein X¹, X², and X³ are 2′-deoxyribonucleotides.

34. The oligonucleotide of any one of claims 1-33, wherein X² comprises a cytosine or 5-methylcytosine nucleobase.

35. The oligonucleotide of claim 34, wherein X² comprises a cytosine nucleobase.

36. The oligonucleotide of any one of claims 1-35, wherein the oligonucleotide further comprises a 5′-cap structure.

37. The oligonucleotide of any one of claims 1-36, wherein the oligonucleotide comprises at least one alternative nucleobase.

38. The oligonucleotide of any one of claims 1-37, wherein m is 5 to 25.

39. The oligonucleotide of any one of claims 1-38, wherein n is 5 to 25.

40. The oligonucleotide of any one of claims 1-39, wherein the activity region is at least 70%, at least 75%, at least 80%, at least 85%, at least 90%, or at least 95% complementary to a target mRNA.

41. The oligonucleotide of any one of claims 1-40, wherein the activity region is complementary to a target mRNA comprising a single nucleotide polymorphism (SNP) associated with a disease or disorder.

42. The oligonucleotide of claim 1-41, wherein the target mRNA encodes a protein comprising a pathogenic amino acid resulting from the SNP.

43. The oligonucleotide of any one of claims 1-42, wherein the oligonucleotide is capable of effecting an ADAR-mediated adenosine to inosine alteration of an adenosine in a target mRNA, wherein X² aligns with the adenosine in the target mRNA to be altered to an inosine.

44. A complex of two oligonucleotides of any one of claims 1-43.

45. The complex of claim 44, wherein the two oligonucleotides are the same.

46. The complex of claim 44 or claim 45, wherein the complex comprises a double-stranded region formed by the duplex regions of the two oligonucleotides.

47. The complex of any one of claims 44-46, comprising a target mRNA.

48. The complex of claim 47, wherein the activity region hybridizes to the target mRNA.

49. A method of editing a target polynucleotide, comprising contacting the target polynucleotide with the oligonucleotide of any one of claims 1-43 or the complex of any one of claims 44-48, thereby editing the polynucleotide.

50. The method of claim 49, wherein the target polynucleotide is contacted with the oligonucleotide in a cell.

51. The method of claim 50, wherein the cell endogenously expresses ADAR.

52. The method of claim 51, wherein the ADAR is a human ADAR.

53. The method of claim 51, wherein the ADAR is human ADAR1.

54. The method of claim 51, wherein the ADAR is human ADAR2.

55. The method of any one of claims 49-54, wherein the cell is selected from eukaryotic cell, a mammalian cell, and a human cell.

56. The method of any one of claim 55, wherein the cell is in vivo.

57. The method of any one of claim 55, wherein the cell is ex vivo.

58. A method of treating a disease or disorder associated with a single nucleotide polymorphism (SNP) in a subject in need thereof, comprising administering to the subject the oligonucleotide of any one of claims 1-43 or the complex of any one of claims 44-48.

59. The method of claim 58, wherein the oligonucleotide or the complex is capable of effecting an ADAR-mediated adenosine to inosine alteration of the SNP associated with the disease or disorder, thereby treating the disease or disorder.

60. The method of any one of claims 58-59, wherein the subject is a human subject.

61. The method of any one of claims 58-60, wherein the target mRNA encodes a protein comprising a pathogenic amino acid resulting from the SNP.

62. The method of claim 61, wherein the adenosine to inosine alteration substitutes the pathogenic amino acid with a wildtype amino acid.

63. The method of claim 61, wherein the adenosine is substituted for another nucleotide that replaces the pathogenic amino acid with an amino acid that confers the substantially similar protein activity as the wildtype amino acid.

64. The method of claim 63, wherein adenosine is substituted for another nucleotide that replaces the pathogenic amino acid with an amino acid that confers restored or modulated function as compared to a protein comprising the pathogenic amino acid.
